SEQUENCE LISTING (63 sequences)

SEQ ID NO: 1 - protein, 5 aa, Synthetic construct
GSGSG

SEQ ID NO: 2 - protein, 19 aa, Synthetic construct
MVVQHNLRAMNSNRMLGIT

SEQ ID NO: 3 - protein, 20 aa, Synthetic construct
KSTEKLSSGYKINRAADDAA

SEQ ID NO: 4 - protein, 19 aa, Synthetic construct
  VARIANT 14: note = X is S or K
DDAAGLTISEKMRXQIRGL

SEQ ID NO: 5 - protein, 35 aa, Synthetic construct
  VARIANT 30: note = X is S or K
KSTEKLSSGYKINRAADDAAGLTISEKMRXQIRGL

SEQ ID NO: 6 - protein, 29 aa, Synthetic construct
QTAEGALTEVHDMLQRMNELAVQAANGTN

SEQ ID NO: 7 - protein, 17 aa, Synthetic construct
LGAVQNRLEHTINNLDN

SEQ ID NO: 8 - protein, 26 aa, Synthetic construct
ENTTAAESQIRDTDMATEMVKYSNNN

SEQ ID NO: 9 - protein, 24 aa, Synthetic construct
LAQAGQSMLAQSNQANQGVLSLLG

SEQ ID NO: 10 - protein, 447 aa, Synthetic construct
EVQLQQSGPVLVKPGASVKMSCKASGYTFTDYYMNWVIQSQGKRLEWIGVINPYNGVKIYNQKFKGKATLTVDKSSSTAYMELNSLTSEDSAVYYCAWDDGYYGNYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, Synthetic construct
DIVMTQTPLTLSVTIGQPASISCKSSQSLLDSDGKTYLNWLLQRPGQSPKRLIYLVSKLDSGVPDRFTGSGSGTDFTLKISRMEAEDLGVYYCWQGTHFPHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 1344 bp, Synthetic construct
  source 1..1344: mol_type = other DNA
gaggtccaactccaacagtctggacctgtgctggtgaagcctggagcctctgtgaagatgagttgtaaggcatctggctacaccttcacagactactatatgaactgggtgattcagagccagggcaagagattggagtggattggagtgataaacccatacaatggagtgaagatttacaaccagaagttcaagggcaaggctaccctgacagtggacaagtcctccagcacagcctatatggaactgaactccctgacctctgaggactctgctgtctactactgtgcctgggatgatggctactatggcaactactggggacaaggcaccaccctgacagtgtccagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacgtgcgtggtggtggacgtgagccacgaagaccccgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 13 - DNA, 660 bp, Synthetic construct
  source 1..660: mol_type = other DNA
gacattgtgatgacccagacaccactgaccctgtctgtgaccattggacaacctgccagcatctcctgtaagtccagccagtccctgctggactctgatggcaagacctacctgaactggctgctccaaagacctggacaaagcccaaagagactgatttacctggtgagcaaactggactctggagtgcctgacaggttcacaggctctggctctggcacagacttcaccctgaaaatcagcaggatggaggctgaggacctgggagtctactactgttggcagggcacccactttccacacacctttggaggaggcaccaaattggagattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - protein, 8 aa, Synthetic construct
GYTFTDYY

SEQ ID NO: 15 - protein, 8 aa, Synthetic construct
INPYNGVK

SEQ ID NO: 16 - protein, 10 aa, Synthetic construct
AWDDGYYGNY

SEQ ID NO: 17 - protein, 11 aa, Synthetic construct
QSLLDSDGKTY

SEQ ID NO: 18 - protein, 9 aa, Synthetic construct
WQGTHFPHT

SEQ ID NO: 19 - protein, 24 aa, Synthetic construct
GSGSGMVVQHNLRAMNSNRMLGIT

SEQ ID NO: 20 - protein, 25 aa, Synthetic construct
GSGSGKSTEKLSSGYKINRAADDAA

SEQ ID NO: 21 - protein, 24 aa, Synthetic construct
  VARIANT 19: note = X is S or K
GSGSGDDAAGLTISEKMRXQIRGL

SEQ ID NO: 22 - protein, 40 aa, Synthetic construct
  VARIANT 35: note = X is S or K
GSGSGKSTEKLSSGYKINRAADDAAGLTISEKMRXQIRGL

SEQ ID NO: 23 - protein, 471 aa, Synthetic construct
MVVQHNMQAMNANRMLNVTTLTEVHSMLQRMNELAVQASNGMVVQHNMTAANANRMGETHSILQRMNELATQAANMVVQHNLTAMNANRQLVGTTGMVVQHNMQAANANRMLGITSVHSMLQRMNELAVQAASNGTNSMVVQHNMQAANANRMLNVTTLTEVHSMLQRMNELATQSANGLTEVHSMLQRMNELAVQSSNGDMAEEMVEYSKNNILAQAGQSMLAQANQSMAEEMVNYSKNNILAAQAGQSMLAQANQMAKEMVNYSKNNILAQAGQSMLAQANDMAEEMVTYSKNNILAQAGQSMLAQANQMVVQHNLRAMNSNRMLGITQSAQRSLLGAVQNRLEHTINNNEAHSILQRMNELAVQGANDVEYSKNNILAQAGQSMLAQANQMVVQHNLRAMNSNRMLSITQDMATEMVKFSNSNILAQAGQMVVQHNLRAMNANRMLGITTEVHDMLQRMNELAVKAAN

SEQ ID NO: 24 - protein, 20 aa, Synthetic construct
MVVQHNMQAMNANRMLNVTT

SEQ ID NO: 25 - protein, 21 aa, Synthetic construct
LTEVHSMLQRMNELAVQASNG

SEQ ID NO: 26 - protein, 15 aa, Synthetic construct
MVVQHNMTAANANRM

SEQ ID NO: 27 - protein, 19 aa, Synthetic construct
GETHSILQRMNELATQAAN

SEQ ID NO: 28 - protein, 453 aa, Synthetic construct
EVQLQQSGPVLVKPGASVKMSCKASGYTFTDYYMNWVIQSQGKRLEWIGVINPYNGVKIYNQKFKGKATLTVDKSSSTAYMELNSLTSEDSAVYYCAWDDGYYGNYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 29 - protein, 219 aa, Synthetic construct
DIVMTQTPLTLSVTIGQPASISCKSSQSLLDSDGKTYLNWLLQRPGQSPKRLIYLVSKLDSGVPDRFTGSGSGTDFTLKISRMEAEDLGVYYCWQGTHFPHTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 30 - DNA, 1362 bp, Synthetic construct
  source 1..1362: mol_type = other DNA
gaggtccaactccaacagtctggacctgtgctggtgaagcctggagcctctgtgaagatgagttgtaaggcatctggctacaccttcacagactactatatgaactgggtgattcagagccagggcaagagattggagtggattggagtgataaacccatacaatggagtgaagatttacaaccagaagttcaagggcaaggctaccctgacagtggacaagtcctccagcacagcctatatggaactgaactccctgacctctgaggactctgctgtctactactgtgcctgggatgatggctactatggcaactactggggacaaggcaccaccctgacagtgtccagtgccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 31 - DNA, 660 bp, Synthetic construct
  source 1..660: mol_type = other DNA
gacattgtgatgacccagacaccactgaccctgtctgtgaccattggacaacctgccagcatctcctgtaagtccagccagtccctgctggactctgatggcaagacctacctgaactggctgctccaaagacctggacaaagcccaaagagactgatttacctggtgagcaaactggactctggagtgcctgacaggttcacaggctctggctctggcacagacttcaccctgaaaatcagcaggatggaggctgaggacctgggagtctactactgttggcagggcacccactttccacacacctttggaggaggcaccaaattggagattaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 32 - protein, 34 aa, Synthetic construct
GSGSGQTAEGALTEVHDMLQRMNELAVQAANGTN

SEQ ID NO: 33 - protein, 22 aa, Synthetic construct
GSGSGLGAVQNRLEHTINNLDN

SEQ ID NO: 34 - protein, 31 aa, Synthetic construct
GSGSGENTTAAESQIRDTDMATEMVKYSNNN

SEQ ID NO: 35 - protein, 29 aa, Synthetic construct
GSGSGLAQAGQSMLAQSNQANQGVLSLLG

SEQ ID NO: 36 - protein, 75 aa, Synthetic construct
MVVQHNLQAMNSNRMLGITQKTASKSTEKLSSGYAINRAADNAAGLAISEKMRKQIRGLTQASTNAEDGISSVQT

SEQ ID NO: 37 - protein, 75 aa, Synthetic construct
MVVQHNLRAMNSNRMLGITQGSLNKSTEKLSSGYKVNRAADDAAGLSISEKMRKQIRGLSQASLNAEDGISAVQT

SEQ ID NO: 38 - protein, 75 aa, Synthetic construct
MVVQHNLRAMNANRMLGITNSGLSKSSEKLSSGYKVNRAADDAAGLSISEKMRKQIRGLTQASVNAEDGISAVQT

SEQ ID NO: 39 - protein, 75 aa, Synthetic construct
MVVQHNLRAMNSNRMLSITQGTLNKSTEKLSSGYKVNRAADDAAGLSISEKMRKQIRGLTQASLNAEDGISAVQT

SEQ ID NO: 40 - protein, 75 aa, Synthetic construct
MVVQHNLTAMNANRQLGVTTSTQAKSSEKLSSGYRINRAGDDAAGLKISEKMRSQIRGLNKASSNAQDGVSLIQT

SEQ ID NO: 41 - protein, 75 aa, Synthetic construct
MVIQHNLTAMNSNRQLGVITSGQAKSSEKLSSGYRINRAADDAAGLKISEKMRSQVRGLNRASTNAQDGISLIQT

SEQ ID NO: 42 - protein, 75 aa, Synthetic construct; identical to SEQ ID NO: 37

SEQ ID NO: 43 - protein, 75 aa, Synthetic construct
MVVQHNMQAMNANRMLNVTTGQQAKSTEKLSSGYRINRAADDAAGLTISEKMRKQIRGLDQASTNAQDGVSSVQT

SEQ ID NO: 44 - protein, 75 aa, Synthetic construct
MVVQHNMTAANANRMLGITTSAQAKSSEKLSSGYKINRAADDAAGLTISEKMRSQVRGLNKASDNAEDGVSLIQV

SEQ ID NO: 45 - protein, 75 aa, Synthetic construct
MVVQHNLTAMNANRQLGVTTGAQAKSSEKLSSGYKINRAADDAAGLTISEKMRSQVRGLNKASDNAQDGVSLIQV

SEQ ID NO: 46 - protein, 75 aa, Synthetic construct
MVVQHNMQAANANRMLGITSGAQAKSTEKLSSGYKINRAADDAAGLTISEKMRKQIRGLDKASSNAQDGVSSVQT

SEQ ID NO: 47 - protein, 75 aa, Synthetic construct; identical to SEQ ID NO: 46

SEQ ID NO: 48 - protein, 75 aa, Synthetic construct
MVVQHNMQAANANRMLNVTTSAQSKSTEKLSSGYRINRAADDAAGLTISEKMRKQIKGLDRASTNAEDGVSAVQT

SEQ ID NO: 49 - protein, 75 aa, Synthetic construct
MVVQHNMQAANANRMLNVTTSAQSKSTEKLSSGYKINRAADDAAGLSISEKMRKQIKGLDRASTNAQDGVSCVQT

SEQ ID NO: 50 - protein, 75 aa, Synthetic construct
MVVQHNMTAMNANRMLGVTTSAQAKSSEKLSSGYRINRAGDDAAGLTISEKMRSQIRGLNKASDNAQDGISLIQV

SEQ ID NO: 51 - protein, 75 aa, Synthetic construct
MVVQHNMSAMNANRNLGVTTGMQAKSSEKLSSGYKINRAADDAAGLSISEKMRSQIRGLNKASDNAQDGISLIQT

SEQ ID NO: 52 - protein, 75 aa, Synthetic construct
MRIQHNIMAMNAYRNYNNNNKSLSGNLEKLSSGYKINRAGDDAAGLAISEKMRAQITGLDAAQKNVKDGISLVKT

SEQ ID NO: 53 - protein, 75 aa, Synthetic construct
MRIQHNIAALNSYRNLSGNNNAVSKNLEKLSSGYRINRAGDDAAGLAISEKMRAQITGLKTAQKNANDGISLVQT

SEQ ID NO: 54 - protein, 75 aa, Synthetic construct
MKINQNISAVIVNDQLLRNENSVAASVQRLSSGLKFNNAKDNPSGMAISFKMQAQINALNRASLNATDGTSMVET

SEQ ID NO: 55 - protein, 74 aa, Synthetic construct
VINTNSLSLLTQNNLNKSQSSLSSAIERLSSGLRINSAKDDAAGQAIANRFTANIKGLTQASRNANDGISVAQT

SEQ ID NO: 56 - protein, 74 aa, Synthetic construct
VINTNSLSLLTQNNLNKSQSSLSSAIERLSSGLRINSAKDDAAGQAIANRFTSNIKGLTQASRNANDGISIAQT

SEQ ID NO: 57 - protein, 15 aa, Synthetic construct
STEKLSSGYKVNRAA

SEQ ID NO: 58 - protein, 15 aa, Synthetic construct
STEKLSSGYKINRAA

SEQ ID NO: 59 - protein, 15 aa, Synthetic construct
AKSSEKLSSGYRINR

SEQ ID NO: 60 - protein, 15 aa, synthetic construct
NLEKLSSGYKINRAG

SEQ ID NO: 61 - protein, 15 aa, synthetic construct
STEKLSSGYRINRAA

SEQ ID NO: 62 - protein, 15 aa, synthetic construct
SSEKLSSGYRINRAG

SEQ ID NO: 63 - protein, 15 aa, Synthetic construct
STEKLSSGYAINRAA

(19) United States
(12) Patent Application Publication
(10) Pub. No.: US 2026/0140112 A1
(43) Pub. Date: May 21, 2026

(54) FLAGELLIN EPITOPE PEPTIDES AND USES THEREOF

(71) Applicant: THE UAB RESEARCH FOUNDATION, Birmingham, AL (US)

(72) Inventors: Charles O. Elson, Birmingham, AL (US); Lennard Wayne Duck, Birmingham, AL (US); Rodney G. King, Birmingham, AL (US); Qing Zhao, Birmingham, AL (US)

(73) Assignee: THE UAB RESEARCH FOUNDATION, Birmingham, AL (US)

(21) Appl. No.: 19/121,177

(22) PCT Filed: Oct. 19, 2023
(86) PCT No.: PCT/US2023/077245

§ 371 Date: Apr. 15, 2025

Related U.S. Application Data
(60) Provisional application No. 63/380,165, filed on Oct. 19, 2022.

(51) Int. Cl. G01N 33/564; C07K 14/195; C07K 16/1267; G01N 33/543; G01N 33/569; G01N 33/68

(52) U.S. Cl. CPC G01N 33/564; C07K 14/195; C07K 16/1267; G01N 33/543; G01N 33/56911; G01N 33/6854; C07K 2317/565; G01N 2333/195

(57) ABSTRACT

Provided herein is a peptide array comprising a plurality of flagellin peptides corresponding to highly conserved peptide regions. For example, the peptide array comprises a plurality of Lachnospiraceae flagellin peptides, which can be selected from a hinge region of Lachnospiraceae flagellin. Also provided is antibody of fragment thereof that binds to one or more of the plurality of the flagellin peptides in the peptide array. The peptide array and antibody are useful in determining an immunosignature from a biological sample from a subject. The immunosignature is useful in diagnosis of an immune-mediated disease, in monitoring progression of an immune-mediated disease, in identifying subjects susceptible to certain treatments, and in monitoring treatment response.

### PRIOR RELATED APPLICATION

This application claims the benefit of and priority to U.S. Provisional Application No. 63/380,165, filed on Oct. 19, 2022, which is hereby incorporated by reference in its entirety.

### SEQUENCE LISTING

The instant application contains a Sequence Listing in XML format. The Sequence Listing, named 035979_1409251seglist.xml, which was created on Oct. 18, 2023, is 63 Kilobytes in size, and is hereby incorporated by reference in its entirety.

### BACKGROUND

Inflammatory bowel diseases such as Crohn's disease and ulcerative colitis are characterized by dysregulated adaptive immune responses to the microbiota in genetically susceptible individuals. However, specificity in these responses vary. For example, certain flagellins have been identified to be immunodominant antigens in Crohn's disease but not ulcerative colitis. Flagellins, components of the hairlike motility flagella that extend from the bacterial cell wall, are potent immune activators and antigens. Flagellins are the only known microbial proteins having three receptors for innate immunity encoded in the host genome, in addition to immunoglobulin and T cell receptors. For this reason, flagellin peptide-specific immunotherapy has been proposed for certain immune-mediated diseases, such as Crohn's disease. However, early diagnosis of an immune-mediated disease and distinguishing those subjects responsive to flagellin peptide-specific immunotherapy has proven difficult to date.

### SUMMARY

Provided herein is a peptide array comprising a plurality of flagellin peptides corresponding to highly conserved peptide regions. For example, the peptide array comprises a plurality of Lachnospiraceae flagellin peptides, which can be selected from a hinge region of Lachnospiraceae flagellin. The peptides in the array can be attached to a substrate in an addressable format. Optionally the substrate is a solid support such as a slide, dish, membrane, or a bead. Specific peptides of the plurality can be attached to a specific location on a slide or dish or can be attached to a specific type of bead. The specific type of bead optionally contains a specific distinguishable detectable label, and a specific distinguishable size, or both a specific distinguishable detectable label, has a specific distinguishable size. Thus, binding to a specific peptide can be discerned by detecting binding to one or more beads having one or more particular, distinguishable characteristics.

The peptide array can be used in methods for identifying an immunosignature in a subject with or suspected of having an immune-mediated disease or in a subject at risk of an immune-mediated disease by contacting a biological sample from the subject with the peptide array under conditions that allow binding of antibodies in the biological sample with the plurality of Lachnospiraceae peptides in the peptide array and detecting the level of binding of antibodies (e.g., IgG) in the biological sample with the plurality of Lachnospiraceae peptides in the peptide array to form an immunosignature for the subject. The subject's immunosignature can then be compared with an immunosignature of one or more control subjects, wherein the one or more control subjects are known to be either negative for immune-mediated diseases or positive for a specific immune-mediated disease. The level of binding can be quantified by use of a control antibody or fragment thereof in known amounts, wherein one or more amounts of a control antibody or fragment thereof binds to one or more of the plurality of the flagellin peptides in the peptide array. Thus, provided herein is a method of diagnosing in a subject an immune-mediated disease (e.g., Crohn's disease) or identifying a subject at risk of developing an immune-mediated disease (e.g., Crohn's disease) marked by certain flagellin antigens. Also provided herein is an antibody or fragment thereof that binds one or more peptides in the plurality of peptides in the array. The antibody described herein can be used as a control antibody at specific concentrations in order to quantify the amount of bound antibody in a sample.

Further provided are methods of treating a subject with an immune-mediated disease (e.g., Crohn's disease). Such a method of treatment can include the steps of identifying an immunosignature in a subject with or suspected of having an immune-mediated disease or in a subject at risk of an immune-mediated disease by contacting a biological sample from a subject with the peptide array; associating the immunosignature with an immune-mediated disease in the subject, and administering a therapeutic agent to the subject having the immune-mediated disease. Such a method of treatment can include determining responsivity of a subject to the therapeutic agent by obtaining a first biological sample from the subject prior to administration of a therapeutic agent; identifying a first immunosignature for a subject by contacting the first biological sample from the subject with the peptide array; administering to the subject an effective amount of the therapeutic agent; obtaining a second biological sample from the subject following administration of the therapeutic agent; identifying a second immunosignature for the subject by contacting the second biological sample from the subject with the peptide array; and comparing the first and second immunosignatures. Subsequently, the therapeutic agent is administered to the subject if the second immunosignature is more similar to the immunosignature of one or more control subjects known to negative for immune-mediated diseases as compared to the first immunosignature. If the second immunosignature is more similar to the immunosignature of one or more control subjects known to be positive for immune-mediated diseases as compared to the first immunosignature, then the dosage regimen of the therapeutic agent is used or a different therapeutic agent is administered to the subject subsequent to the comparing step. By way of example, the immune-mediated disease can be an inflammatory bowel disease such as Crohn's disease or ulcerative colitis and the therapeutic agent can be a polypeptide comprising one of more flagellin T cell receptor epitopes.

The details of one or more embodiments are set forth in the description below. Other features, objects, and advantages will be apparent from the description and from the claims.

### BRIEF DESCRIPTION OF THE DRAWINGS

FIGS. 1A-E show that Lachnospiraceae flagellin peptide microarray identifies key IgG binding epitopes in CD patients. (A) Schematic strategy of the flagellin peptide microarray. (B) Heatmap showing serum IgG reactivity for sequential, overlapping fifteen-residue flagellin peptides (108 peptides from each of the 19 different flagellins) in CD (n=135), HC (n=42), and UC (n=88) subjects. Sera were diluted at 1:100. Rows (subjects) were clustered using Euclidean distance and complete linkage applied to log-transformed data. Columns (peptides) are sorted by peptide position (and include peptides from up to 19 different flagellins per position). Cohort groups are indicated by colored dots on the right margin. Peptide starting position (relative to the N-terminus) is indicated on the bottom. For each 15-mer segment, per-residue information content is computed at each position across all flagellins (except L. CBir11 Fla and L. CBir66 Fla) and summated to indicate sequence conservation, shown as color. Although all peptides were used for the clustering, only peptides up to and including 121-136 are depicted in this heatmap. (C) 3D structure of L. A4 Fla2 as a representative flagellin showing the location of the hinge region. (D) Sequence alignment of flagellins (SEQ ID NOs: 36-56) included in the peptide microarray plus S. dublin FliC and E. coli FliC. Sequences were aligned in BioEdit using the ClustalW alignment feature. Flagellin D0 and D1 domains in the N-term are indicated by the color bars on top, whereas the hinge region is marked by a box. (E) Motif discovery of the flagellin hinge region.

FIGS. 2A-M show serum IgG multi-flagellin reactivity in CD patients is driven by reactivity to the dominant Lachnospiraceae flagellin peptide epitope. (A) Schematic strategy of the flagellin peptide cytometric bead array. (B and C) Gating strategies of the flagellin peptide cytometric bead array. (D) Sera from CDhigh (n=39), CDlow (n=126), UC (n=181), and HC (n=95) subjects in the UAB cohort were probed against the flagellin peptide cytometric bead array. Representative flow plots of serum IgG reactivity to flagellin peptide epitopes in each group are shown. The red box highlights the hinge region peptides. (E) Histogram overlay of serum IgG reactivity to Lachnospiraceae D0-1N p25-59 in 3 representative CDhigh, CDlow, UC, and HC, respectively. (F-M) Epitope-specific IgG concentration in the serum of CDhigh. CDlow, UC, and HC subjects to indicated flagellin peptide epitopes. Data are presented as means±SEM and analyzed with nonparametric Kruskal-Wallis test. *P<0.05, **P<0.01, ***P<0.001, ****P<0.0001.

FIGS. 3A-G show Lachnospiraceae hinge peptide is the dominant B cell epitope in pediatric CD patients and predicts the development of disease complications. (A) Sera of CD patients (grouped by disease behavior at 3-year follow-up using the Montreal classification, n=250 for B1 (inflammatory), n=142 for B2 (stricturing) or B3 (penetrating)) and non-IBD controls (n=72) from the RISK cohort were probed against the microbiota protein microarray. Mean fluorescent intensity (MFI) of serum IgG specific for indicated flagellin antigens is presented as means±SEM and analyzed with nonparametric Kruskal-Wallis test. *P<0.05, **P<0.01, ***P<0.001, ****P<0.0001. (B) Heatmap of log 10 IgG response (color) on the microbiota protein microarray for 21 flagellins (rows) and 392 individuals with CD in the RISK cohort (columns). Rows and columns were hierarchically clustered using Euclidean distance on the log data, average linkage and optimal leaf ordering maximizing the sum of similarity between every leaf and all other leaves in the adjacent cluster. Montreal disease classification at follow-up (B2 and B3) is indicated below the heatmap, with unmarked individuals being B1. The bar graph at the bottom indicates the number of positive flagellin responses for each individual, where a positive response is two standard deviations above the mean (non-log transformed) of a non-IBD control cohort (n=72, not shown) per flagellin. (C and D) Summary of multi-flagellin reactivity in CD patients with B1 or B2/3 behavior at follow-up. (E-G) Sera of CD patients (grouped by disease behavior at 3-year follow-up, n=249 for B1, n=140 for B2/3) and non-IBD controls (n=72) from the RISK cohort were probed against the flagellin peptide cytometric bead array. Epitope-specific IgG concentration in different groups to indicated flagellin peptide epitopes is shown. Data are presented as means±SEM and analyzed with nonparametric Kruskal-Wallis test. *P<0.05, **P<0.01, ****P<0.0001.

FIGS. 4A-D show serum IgG to Lachnospiraceae hinge peptide strongly correlates with multi-flagellin reactivity in CD. (A) Heatmaps representing correlation coefficients between serum IgG anti-flagellin reactivity (log 10 MFI from microbiota protein microarray; rows) and serum IgG anti-peptide reactivity (hyperbolic arcsin MFI from flagellin peptide cytometric bead array; columns). Four separate heatmaps are shown for CD (n=148), HC (n=86), and UC (n=169) participants from the UAB cohort and CD (n=414) participants from the RISK cohort, respectively. Color corresponds to Pearson correlation coefficient indicated in the bar scale. Dots indicate correlations that were below Bonferroni-corrected p-value of 0.01 (based on 21 flagellins, 8 peptides, 4 groups=672 tests) testing the hypothesis of no correlation against the alternative that there is a non-zero correlation. Rows (flagellins) are sorted based on the correlation for peptide D0-1N p25-59 in CD patients in the RISK cohort, as indicated by the triangle. (B) Hyperbolic arcsin MFI of serum IgG anti-Lachnospiraceae D0-1N p25-59 and D1C p391-407 of individual CD, HC, and UC participants from the UAB cohort and CD participants from the RISK cohort obtained from the flagellin peptide cytometric bead array was plotted against reactivity (log 10 MFI) to R. intestinalis Fla1 and E. coli FliC obtained from the microbiota protein microarray, respectively. Least squares regression was performed, with the correlation line in red and R value shown for each plot. (C and D) The value of hinge-specific IgG obtained from the flagellin peptide cytometric bead array is plotted on X axis, and the number of flagellins bound by sera in the same individual CD patients on the microbiota protein microarray on the Y axis. Data from the UAB cohort and the RISK cohort are presented as indicated.

FIGS. 5A-C show that serum IgG reactivity to Lachnospiraceae flagellin hinge peptide is a homeostatic response in most infants. (A) Sera of healthy and non-IBD control infants at 0 month (cord blood, CB), 6 months, and 12 months of age (with all three timepoints) from the Uganda cohort (n=76) and the US (n=10)/Sweden (n=36) cohort were probed against the flagellin peptide cytometric bead array. Concentration of serum IgG specific to Lachnospiraceae flagellin D0-1N p25-59 in different groups is shown. Each dot represents an individual and responses of the same individual at different timepoints are linked with the grey lines. The means of serum IgG response to Lachnospiraceae flagellin D0-1N p25-59 of CD patients in the UAB (broken line) and RISK (solid line) cohort are plotted. Data are presented as means±SEM and analyzed with repeated measures ANOVA and Friedman test. ****P<0.0001. (B and C) Sera of healthy and non-IBD control infants at indicated timepoints (including individuals not represented in all three timepoints) from the Uganda cohort (n=92) and the US (n=16)/Sweden (n=54) cohort were probed against the flagellin peptide cytometric bead array. IgG concentration specific to Lachnospiraceae D0N p25-44 and DIN p41-59 sub-epitopes in different groups is shown, respectively. Data are presented as means±SEM and analyzed with two-way ANOVA and Tukey's multiple comparisons test. *P<0.05, ***P<0.001, ****P<0.0001.

FIG. 6 shows the design for an array panel. Top left shows the flow cytometric read-out for the flagellin consensus peptides (bottom left) and for the microbiota antigen protein panel (right).

FIG. 7 is a graph showing the number of IgG anti-flagellin positive sera on the flagellin protein microarray on the Y axis and the IgG anti-hinge peptide in pg/ml on the X axis.

FIG. 8 is a heat map of a flagellin protein microarray illustrating sera from subjects with Crohn's disease (CD high and CD low) using the flagellin protein microarray in which flagellin proteins are printed on nitrocellulose coated slides.

FIG. 9 shows the work-flow for rapid recombinant monoclonal antibody generation using IgClic technology.

FIG. 10A shows a predicted three-dimensional structure for Flagellin 2 (Fla2).

FIG. 10B shows the mean fluorescent intensity (MFI) for binding of mAb IgG F6 to multiple Lachnospiraceae flagellin (peptides on the peptide array (p25-41) (SEQ ID Nos: 57-63)) as shown in FIG. 2. mAB IgG F6 did not bind Salmonella Fli C or E. coli Fli C above background, indicating that the antibody specifically binds Lachnospiraceae flagellin peptides.

FIG. 10C is a graph showing the MFI for mAb IgG F6 binding of various flagellins using the flagellin protein microarray and to two proteobacterial flagellins from S. Dublin and E. coli. The results confirm that the mAb F6 binds not only to selected peptide epitopes as shown in FIG. 10B but also to native flagellin proteins expressing this epitope. Mean and individual triplicate measurements of mean fluorescent intensity are shown for each flagellin.

FIG. 11 is a schematic demonstrating various schemes for engineering antibodies and antibody fragments having the variable heavy (VH) and variable light (VL) sequences of the original antibody.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Lachnospiraceae bacteria are gram positive, anaerobic, fermentative, and mucus-associated bacteria found in the ileum and right colon. These bacteria are generally considered beneficial commensals in the gut, due to their ability to convert plant polysaccharides into short-chain fatty acids (SCFA), which feed epithelial cells and support the development and maintenance of intestinal regulatory CD4 T cells (Tregs). However, in certain immune-mediated diseases, Lachnospiraceae flagellin is key to immune reactivity driving inflammation. Multi-flagellin reactivity can serve as a harbinger of the onset or presence of an immune-mediated disease of a subject and can indicate lack of responsiveness to treatment modality. Thus, provided herein are peptide arrays and antibodies for use in detecting and quantifying antibodies in a biological sample (e.g., a serum, blood, intestinal sample, or plasma sample) from a subject in order to identify an immunosignature related to Lachnospiraceae flagellin reactivity.

### Peptide Arrays

Provided herein is a peptide array comprising a plurality of Lachnospiraceae flagellin peptides. The Lachnospiraceae flagellin peptides of the plurality are fragments of the hinge region of a Lachnospiraceae flagellin. These peptides correspond to highly conserved regions of the Lachnospiraceae family of flagellins, which are the dominant targets of IgG cell antibodies in subjects with Crohn's disease.

The peptides of the plurality can be used in microarrays, for example, in any immunoassay platform, such as, for example, multiplex or highthroughput immunoassay platforms. Microarrays can comprise a number of distinct agents (e.g., peptides), sometimes referred to as probes, which are immobilized, for example, on a substrate, in specific locations, known as the spots. Each spot usually contains one type of a probe. Individual spots form a two-dimensional grid or array. Linear coordinates of each spot within such grid are used to determine the identity of the probe at that position. Consequently, the identity of a protein that interacts with each probe, sometimes referred to as a target, may be determined based of the specificity of the probe-target interaction. Microarrays of this type are known as ordered arrays or printed arrays. The unambiguous correlation between the identity of the probe and its location on the microarray slide is known as positional encoding.

Alternative microarray formats also exist, in which the identity of a probe cannot be determined from its location. Such microarrays are known as random arrays. An example of a random array is ILLUMINA® BEADARRAY™ in which individual reactive microbeads are randomly placed into wells etched on a microwell array plate. The identity of a probe in random arrays may be determined using bead encoding and subsequent decoding, i.e. each bead carries a unique identifying label. A variety of bead encoding technologies are known in the art.

In some examples, the peptides of the plurality are attached to a substrate, such as a polyethylene/poly (acrylic acid) substrate or a carbon substrate (e.g. a carbon electrode). The substrate can be selected from a planar substrate (e.g., a slide, plate, dish, or membrane, such as a cellulose membrane) or a moveable substrate such as a set of beads. For planar substrates, as described above, the peptides are attached in a pattern, such that each region or spot on the surface presents a specific peptide. Optionally the peptides are attached to beads which are themselves attached to a slide, a plate, a dish, or a membrane, for example. However, the beads can be unattached to an additional surface and can be sortable using, for example, cell sorting systems such as flow cytometry and fluorescent-activated cell sorting (FACS). Each different Lachnospiraceae flagellin peptide of the plurality is optionally attached to a different type of bead. The beads are optionally of various distinguishable sizes (e.g., 3 mm, 4 mm, or 5 mm) and optionally contain various detectable labels (e.g., fluorophores such as Alexa 405, Alexa 647, Alexa 488. Alexa 700, AmCyan, BV421BV510, BV605, BV650, BV711, FITC, Pacific Blue, eFluor 710, GFP, Oregon Green, PE, Texas Red, mCherry, PE-Cy5, PE-Cy5.5, PE-Cy7, and others), allowing for use in a multiplex assay, i.e., an immunoassay that simultaneously measures multiple analytes in a single experiment. Thus, a peptide of a specific sequence is optionally attached to a bead having a first size (e.g., 3 mm, 4 mm, or 5 mm) and/or detectable label (e.g., a fluorophore) and peptides of a different sequence are attached to a bead having a second size and/or detectable label so that the plurality of beads can be sorted according to size and/or label to determine which sequences are bound by antibodies in a test sample. One of skill in the art can select fluorochromes and excitation channels that permit differentiation of the selected detectable labels. By way of example, one of skill in the art could select detectable labels that can be differentiated by fluorescent intensity in the APC/Cy7 channel. Such differentiation permits identification of IgG, IgA, and IgM reactivity to different antigens simultaneously.

The beads are optionally attached to the substrate by a coupling means such as biotin-avidin coupling. Optionally the peptides of the plurality are biotinylated and the beads are coated with avidin. Further the biotin can be coupled to the peptides of the plurality by means of a linker. Linkers are known in the art and include, for example, GS, GSG, GGSG, or GSGSG (SEQ ID NO: 1) linkers. These linkers are optionally repeats of the subunit one or more times. For example, a GS linker can be a GSn linker wherein n is a numerical number being 1, 2, 3, 4, 5, 6, 7, 8, 9, 10 or more.

In some examples, instead of being placed on a solid support, a plurality of beads mas react with the sample and undergo subsequent measurement by an analytical method while suspended in a liquid medium. This format is known as a suspension bead array or a liquid array. Flow cytometry and fluorescence-activated cell sorting (FACS) are used for the screening of individual beads in a suspension bead array.

In some cases, the plurality of peptides are used in a Meso Scale Discovery (MSD) system. The term MSD or MSD system, as used herein, refers to methods incorporating detection and quantification by the MSD ECL detection system (Meso Scale Discovery, Gaithersburg, Md.). An MSD system employs a label for example, a ruthenium (II) tri-bipyridine-(4-methylsulfonate) NHS ester label that emits light upon electrochemical activation (e.g., SULFO-TAG™, Meso Scale Discovery (Gaithersburg, Md.)). ECL measurements can be carried out using screen-printed carbon ink electrodes. These carbon electrodes have 10 times the binding capacity of a polystyrene plate, and are patterned on the bottom of specially designed multi-well or multi-domain multi-well plates, for example, as described in U.S. Pat. No. 7,063,946, which is herein incorporated by reference in its entirety (e.g., MULTI-ARRAY® and MULTI-SPOT® microplates, MSD (Gaithersburg, Md.)). Each well of the plate can have a patterned working electrode comprising a plurality of assay peptides (e.g., 2, 3, 4, 5, 6, 7, 8 or more), approximately in the center of the well, that are exposed regions of electrode surface, which are defined, for example, by a patterned dielectric layer. The dielectric layer can be used to confine small volumes of liquid to specific assay domains. Each well can also have two counter electrodes surfaces (e.g., approximately at two edges of the well). ECL from the ECL labels on the surface of the carbon electrodes can be induced and measured using an imaging plate reader compatible with the MSD system (e.g., SECTOR® Imager 6000 and SECTOR® Imager 2400, MSD (Gaithersburg, Md.)).

The peptides of the plurality are selected from amino acid fragments within the flagellin hinge region and the plurality can comprise two or more (e.g., three or more, four or more, five or more, six or more) peptides. Examples of peptides include those selected from residues 25-59 of the Lachnospiraceae flagellin. For example, the plurality optionally comprises three or more peptides having amino acid sequences selected from Domains D0 25-44, D1 41-59 and D0-D1 25-59 of the Lachnospiraceae flagellin. Exemplary sequences for peptides in the plurality include peptides having amino acid sequences of SEQ ID NO: 3, SEQ ID NO: 4, and SEQ ID NO: 5. In addition, the plurality of peptides can further comprise one or more peptides selected from Domains D0 1-19, D1 74-102, D1 391-407, D1-D0 410-435, and D0 437-460 of the Lachnospiraceae flagellin. Exemplary sequences for Domain D0 1-19, D1 74-102, D1 391-407, D1-D0 410-435, and D0 437-460 include the amino acid sequences of SEQ ID NO: 2, SEQ ID NO: 6, SEQ ID NO: 7, SEQ ID NO: 8, and SEQ ID NO: 9. Similarly, the sequences may vary such that the amino acid sequences of the plurality optionally comprises amino acid sequences that are at least 85%, 90%, 95%, or 99% identical to SEQ ID NO: 3, SEQ ID NO: 4, and SEQ ID NO: 5 and optionally further comprises one or more amino acid sequences that are at least 85%, 90%, 95%, or 99% identical to SEQ ID NO: 2, SEQ ID NO: 6, SEQ ID NO: 7, SEQ ID NO: 8, and SEQ ID NO: 9. Such sequences optionally include truncations, deletions and/or substitutions, but, taken together, the plurality of peptides are sufficient to bind antibodies directed to one or more peptides within the flagellin hinge region.

Peptides of the array can be synthesized, optionally with a linker, using various means of peptide synthesis. Examples of protein synthesis include solution-phase techniques, Merrifield solid-phase synthesis, and in situ synthesis (e.g., SPOT, particle-based synthesis, and photolithographic methods). Synthesized peptides can be attached to the substrate using biological (e.g., using a binding pair like biotin and avidin or streptavidin), physical, or chemical immobilization means. Optionally, the synthesized peptide is then biotinylated by adding a biotin moiety to either the N- or C-terminus of each peptide, optionally with an intervening linker sequence. The substrate, e.g., beads, coated with avidin or streptavidin are then contacted with the biotinylated peptides under conditions that permit attachment of the biotinylated peptides to the avidin on the substrate. Specific peptides can be attached to beads having a detectable size (e.g., 3 mm, 4 mm, or 5 mm beads) and/or a detectable label to produce a peptide array suitable for sorting and multiplex analysis.

Optionally, peptides can be synthesized with a C-terminal glycine-serine-cysteine GSC linker, then printed on aminosilane coated glass slides that have been pre-activated with sulfo-SMCC such that the maleimide-activated surface reacts with the peptide terminal cysteine. Slides can be blocked (e.g., in PBS, 3% BSA, 0.05% Tween 20, 0.014% (3-mercaptohexanol for 1 h at 25° C.) to block any unreacted linker on the slide surface. Bound antibodies from a biologic fluid can be detected with fluorochrome labeled anti-IgG, anti-IgA, and anti-IgM using a laser imager.

### Antibodies

The plurality of peptides in the peptide array are selected to bind antibodies directed to the hinge region of Lachnospiraceae flagellin. The antibody optionally binds most Lachnospiraceae flagellins but does not bind proteobacterial flagellins like Salmonella FliC or E. coli FliC. For purposes of quantification of the amount of bound antibody in a biological sample, a standard antibody or antibody fragment that binds the hinge region of Lachnospiraceae flagellin is described herein. Thus, provided herein is an antibody or fragment thereof that binds to one or more of the plurality of flagellin peptides in the peptide array. Optionally, the antibody binds one or more of Domains D0 25-44, D1 41-59 and D0-D1 25-59 of the Lachnospiraceae flagellin. As used throughout, the term antibody encompasses, but is not limited to, a nanobody, a whole immunoglobulin (i.e., an intact antibody) of any class, including polyclonal and monoclonal antibodies, as well as fragments of antibodies that retain the ability to bind their specific antigens. Also useful are conjugates of antibody fragments and antigen-binding proteins (single chain antibodies) as described, for example, in U.S. Pat. No. 4,704,692, the contents of which are hereby incorporated by reference in their entirety.

The antibodies are optionally engineered chimeric antibodies. For example, CDRs from a mouse, rat, or rabbit antibody are contained within a human framework to form a humanized antibody or a fragment thereof. The heavy chain of the antibody or fragment thereof optionally comprises CDR amino acid sequences having at least 85%, 90%, 95%, or 99% identity to GYTFTDYY (SEQ ID NO: 14), INPYNGVK (SEQ ID NO: 15), and AWDDGYYGNY (SEQ ID NO: 16). The light chain of the antibody or fragment thereof optionally comprises CDR amino acid sequences having at least 85%, 90%, 95%, or 99% identity to QSLLDSDGKTY (SEQ ID NO: 17), LVS, and WQGTHFPHT (SEQ ID NO: 18). An exemplary humanized antibody comprises a heavy chain having the amino acid sequence with at least 85%, 90%, 95%, or 99% identity to SEQ ID NO: 10 and a light chain having the amino acid sequence with the amino acid sequence having at least 85%, 90%, 95%, or 99% identity to SEQ ID NO: 11. The humanized antibody that specifically binds the Lachnospiraceae flagellin hinge region optionally comprises a heavy chain with the amino acid sequence of SEQ ID NO: 10 and a light chain with the amino acid sequence of SEQ ID NO: 11.

The antibody can be an IgG, and IgE, and IgM, or an IgA. When the antibody is an IgG it can be of subtype IgG1, IgG2, IgG3, or IgG4.

The antibody is optionally engineered to be a bispecific antibody that binds multiple domains within the hinge region of the Lachnospiraceae. For example, the antibody could bind two domains selected from the group consisting of one or more of Domains D0 25-44, D1 41-59, D0-D1 25-59.

Further provided herein are antibody fragments that bind the hinge region of the Lachnospiraceae flagellin. Such antigen-binding fragments include single-chain variable fragments (scFV) and Fab fragment, which can include the CDRs described herein.

Antibodies disclosed herein can be generated by immunizing mice with flagellin from Lachnospiraceae A4 flagellin and isolating antibody producing B cells from the immunized animal. Provided herein is an exemplary Lachnospiraceae flagellin binding murine IgG2b monoclonal antibody (murine mAB F6 antibody) comprising a heavy chain comprising the amino acid sequence set forth as SEQ ID NO: 27 and a light chain comprising the amino acid sequence set forth as SEQ ID NO: 28. The nucleic acid sequence encoding the heavy chain comprises SEQ ID NO: 29 and the nucleic acid sequence encoding the light chain comprises SEQ ID NO: 30. Also provided is a murine antibody comprises a heavy chain having the amino acid sequence with at least 85%, 90%, 95%, or 99% identity to SEQ ID NO: 27 and a light chain having the amino acid sequence with the amino acid sequence having at least 85%, 90%, 95%, or 99% identity to SEQ ID NO: 28.

The variable region of antibodies produced by the B cells can be sequenced (e.g., using single B cell sequencing technologies) and recombinant DNA constructs (e.g., plasmids) generated for expression in mammalian cell culture (e.g., expression by HEK293 cells). Thus, provided herein are nucleic acids and constructs encoding the antibodies or fragments thereof.

Any of the antibodies provided herein, including human and murine antibodies (e.g., human mAB F6 and murine mAB F6) can be used in any of the methods described herein. Further, any of the antibodies provided herein can be used to detect Lachnospiraceae flagellin in any in vivo or in vitro method comprising detection of Lachnospiraceae flagellin. For example, and not to be limiting, any of the antibodies or fragments thereof provided herein can be used to detect Lachnospiraceae flagellin in Western blots, immunohistochemistry methods, imaging methods etc. In some examples, the antibody is used to detect Lachnospiraceae flagellin in an animal model for inflammatory bowel disease, for example, a murine model of inflammatory bowel disease (e.g., colitis).

### Assay Methods

The peptide array and antibodies described herein can be used to identify an immunosignature in a subject. Bacterial flagellins can be strong stimulators of both innate and adaptive immunity and are an immunodominant antigen in microbiota. Human newborns, for example, have high IgG responses to flagellins, whereas healthy adults have a measurable but low level of serum IgG to flagellins. In subjects with certain immune-mediated diseases, flagellin can be the dominant antigen. Because peptides of Lachnospiraceae family flagellins can be dominant targets of B cell antibodies in a subject with or at risk of developing an immune-mediated disease, the peptide array can be used to identify an immunosignature in a person with or suspected of having an immune-mediated disease or in a person at risk of developing an immune-mediated disease. The immunosignature can serve to diagnose a subject with an immune-mediated disease, can serve to monitor effectiveness of treatment, and can be used to determine receptivity of a subject to specific treatments.

The method comprises contacting a biological sample from the subject with the peptide array under conditions that allow binding of antibodies in the biological sample with the plurality of Lachnospiraceae peptides in the peptide array and detecting the level of binding of antibodies in the biological sample with the plurality of Lachnospiraceae peptides in the peptide array to form an immunosignature for the subject. Optionally, detecting the level of bound antibodies comprises detection of the level of bound IgG, IgA, or IgM or the subtypes of IgG, IgA, or IgM. The immunosignature can include the pattern of binding (e.g., binding to multiflagellin domains), level of binding (e.g., higher levels of binding indicating greater immunoreactivity in the subject), and the type of antibody (e.g., IgG). The immunosignature of the subject being tested can then be compared to one or more control immunosignatures. The one or more control immunosignatures are immunosignatures from subjects known to be either negative for immune-mediated diseases or positive for a specific immune-mediated disease. For example, the immunosignature could be similar to that of one or more healthy subjects or similar to one or more subjects known to have an immune-mediated disease, such as inflammatory bowel disease (e.g., ulcerative colitis or Crohn's disease). By way of example, Lachnospiraceae flagellins are the dominant targets of IgG B cell antibodies in subjects with Crohn's disease and multiflagellin reactivity indicates Crohn's disease with more complications and worse predicted outcome. However, reactivity to one or more peptides of the peptide array is predictive of receptivity to treatment with an anti-flagellin therapeutic agent.

The method optionally further comprises detecting the level of binding of the antibodies in the biological sample with the plurality of flagellin peptides in the peptide array using one or more amounts of a control antibody or fragment thereof as described herein, wherein the antibody binds to one or more of plurality of the flagellin peptides in the array. By using multiple concentrations of an antibody or antibody fragment that binds to the hinge region of Lachnospiraceae flagellin, a standard curve can be generated. The signal of the biological sample (e.g., mean fluorescent intensity) can be quantified by plotting the signal intensity on the standard curve. Alternatively, known amounts of a control polyclonal antibody such as anti-IgG can be bound to the beads, and a standard curve constructed from binding of a secondary tagged anti-immunoglobulin. Unknown samples, being detected with the same anti-IgG, can then be quantified.

Detection of binding can be performed using chemiluminescent, colorimetric, or fluorescence detection. Antibodies (IgG, for example) bound to a set or subset of fluorescent beads, for example, can be detected using a fluorescent detector.

### Methods of Treatment

Provided herein is the use of the peptide array as a companion to accompany treatment of an immune-mediated disease in a subject. The method comprises identifying an immunosignature in a subject with or suspected of having an immune-mediated disease or in a subject at risk of an immune-mediated disease by contacting a biological sample from a subject with the peptide array; associating the immunosignature with an immune-mediated disease in the subject, and administering a therapeutic agent to the subject having the immune-mediated disease. Associating the immunosignature of the sample with an immune-mediated disease entails comparing the immunosignature of the test sample with the immunosignature of one or more control immunosignatures as described above to determine whether the immunosignature of the test sample correlates with a healthy subject or a subject with a known immune-mediated disease, such as an inflammatory bowel disease (e.g., ulcerative colitis or Crohn's disease). Optionally, the therapeutic agent given to the subject is selected to treat the specific immune-mediated disease. For example, when the immunosignature is associated with an immune-mediated disease responsive to an anti-flagellin therapeutic agent, the therapeutic agent administered to the subject is an anti-flagellin therapeutic agent. By way of example, the anti-flagellin therapeutic agent is optionally a polypeptide comprising one of more flagellin T cell receptor epitopes.

For a subject that is largely asymptomatic or is showing minimal signs of an immune-mediated disease but has an immunosignature that is more similar to the immunosignature of a subject with an immune-mediated disease, the subject can be identified to be at risk for developing an immune-mediated disease. Progression of the immunosignature (e.g., more antibody binding) over time could be performed in the subject to monitor progression of the disease. A person of skill in the art would determine whether and when treatment with a therapeutic agent or other intervention is indicated.

Also provided is a method for treating an immune-mediated disease in a subject comprising obtaining a first biological sample from the subject prior to administration of a therapeutic agent; identifying a first immunosignature for a subject by contacting the first biological sample from the subject with the peptide array; administering to the subject an effective amount of the therapeutic agent; obtaining a second biological sample from the subject following administration of the therapeutic agent; identifying a second immunosignature for the subject by contacting the second biological sample from the subject with the peptide array; comparing the first and second immunosignatures; and administering the therapeutic agent to the subject subsequent to the comparing step if the second immunosignature is more similar to the immunosignature of one or more control subjects known to be negative for immune-mediated diseases as compared to the first immunosignature; or modifying the dosage regimen of the therapeutic agent or administering a different therapeutic agent to the subject subsequent to the comparing step if the second immunosignature is more similar to the immunosignature of one or more control subjects known to be positive for immune-mediated diseases, as compared to the first immunosignature. Thus, by acquiring immunosignatures from a subject before and after treatment with a therapeutic agent (e.g., an anti-flagellin therapeutic agent) and determining a shift in the immunosignature toward the immunosignature of a healthy control, the efficacy of the therapeutic agent is confirmed. Conversely, if no change or a shift toward the immunosignature of a subject with an immune-mediated disease occurs, a lack of efficacy is determined. Faced with a lack of efficacy, the person of skill in the art would modify the dosing regime of the therapeutic agent (e.g., by increasing the frequency of dosing or by increasing the amount of the therapeutic agent in a dose), would combine the therapeutic agent with a second therapeutic agent, or would administer a different therapeutic agent to the subject. Thus, an mTOR metabolic inhibitor and/or an AMPK activator (e.g., metformin, troglitazone, pioglitazone, rosiglitazone, resveratrol, quercetin, genistein, epigallocatechin gallate, berberine, curcumin. ginsenoside Rb1, α-lipoic acid and cryptotanshinone), a chemotherapeutic, an immunotherapy, gene therapy, cell transplant therapy, genome editing therapy or other therapeutic agents can be administered to a subject at the same time, prior to, or after administration of a polypeptide comprising one or more flagellin TCR epitopes. Conversely, an mTOR metabolic inhibitor and/or an AMPK activator, a chemotherapeutic, an immunotherapy, gene therapy, cell transplant therapy, genome editing therapy or other therapeutic agents can be administered to a subject instead of a polypeptide comprising one or more flagellin TCR epitopes.

The polypeptide comprising one or more flagellin TCR epitopes comprises one or more microbiota flagellin TCR epitopes. The TCR epitopes can be selected from one or more flagellins selected from the group consisting of Lachnospiraceae, R. inulinivorans, R. hominis, R. faecis, Eubacteria rectale (Agathobacter rectalis), and R. intestinalis. The Lachnospiraceae flagellin is optionally selected from the group consisting of Lachnospiraceae Flax, Lachnospiraceae 14-2, Lachnospiraceae A4, and Lachnospiraceae CBir1. The one or more flagellin TCR epitopes included in the polypeptide are optionally from human microbiota and/or murine microbiota. For details regarding polypeptides comprising one or more flagellin TCR epitopes and for methods of administering the polypeptides to a subject, see International Application Publication No. WO2020/163782, which is incorporated herein in its entirety.

Examples of polypeptides comprising one or more flagellin TCR epitopes include, but are not limited to, polypeptides comprising, consisting of, or consisting essentially of, one or more flagellin TCR epitopes comprising SEQ ID NO: 24 (MVVQHNMQAMNANRMLNVTT). In some methods, the polypeptide further comprises SEQ ID NO: 25 (LTEVHSMLQRMNELAVQASNG). In some methods, the polypeptide further comprises SEQ ID NO:26 (MVVQHNMTAANANRM). In some methods, the polypeptide further comprises SEQ ID NO:27 (GETHSILQRMNELATQAAN).

An example of a multi-epitope polypeptide comprising SEQ ID NO: 24, SEQ ID NO: 25 and SEQ ID NO: 26 and SEQ ID NO: 27 is SEQ ID NO: 23

(SEQ ID NO: 23)
(MVVQHNMQAMNANRMLNVTTLTEVHSMLQRMNELAVQASNGMVV
QHNMTAANANRMGETHSILQRMNELATQAANMVVQHNLTAMNANR
QLVGTTGMVVQHNMQAANANRMLGITSVHSMLQRMNELAVQAASN
GTNSMVVQHNMQAANANRMLNVTTLTEVHSMLQRMNELATQSANG
LTEVHSMLQRMNELAVQSSNGDMAEEMVEYSKNNILAQAGQSMLA
QANQSMAEEMVNYSKNNILAAQAGQSMLAQANQMAKEMVNYSKNN
ILAQAGQSMLAQANDMAEEMVTYSKNNILAQAGQSMLAQANQMVV
QHNLRAMNSNRMLGITQSAQRSLLGAVQNRLEHTINNNEAHSILQ
RMNELAVQGANDVEYSKNNILAQAGQSMLAQANQMVVQHNLRAMN
SNRMLSITQDMATEMVKFSNSNILAQAGQMVVQHNLRAMNANRML
GITTEVHDMLQRMNELAVKAAN).

As used herein, the phrase. T cell, refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), helper T cells, cytotoxic T cells, memory T cells, regulatory T cells, natural killer T cells, combinations thereof, or sub-populations thereof. T cells can be CD4+, CD8+, or CD4+ and CD8+. T cells can also be CD4−, CD8−, or CD4− and CD8−. T cells can be helper cells, for example helper cells of type TH1, TH2, TH3, TH9, TH17, or TFH. T cells can be cytotoxic T cells. Regulatory T cells can be FOXP3+ or FOXP3−. In some cases, the T cell is a CD4+CD25hiCD127lo regulatory T cell. In some cases, the T cell is a regulatory T cell selected from the group consisting of type 1 regulatory (Tr1), TH3, CD8+CD28−, Treg17, and Qa-1 restricted T cells, or a combination or sub-population thereof. In some methods, the polypeptide comprising one or more TCR epitopes activates flagellin-specific CD4+ T cells. In some methods, the polypeptide comprising one or more TCR epitopes activates flagellin-specific CD4+ memory T cells.

As used herein, administer or administration refers to the act of introducing, injecting or otherwise physically delivering a substance as it exists outside the body (e.g., a multi-epitope polypeptide and/or an mTOR metabolic inhibitor) into a subject, such as by mucosal, intradermal, intravenous, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, the term therapeutically effective amount or effective amount refers to an amount of a polypeptide comprising one or more flagellin TCR epitopes, an mTOR metabolic inhibitor or AMPK activator that, when administered to a subject, is effective to treat a disease or disorder either by one dose or over the course of multiple doses. A suitable dose can depend on a variety of factors including the particular polypeptide used and whether it is used concomitantly with other therapeutic agents. Other factors affecting the dose administered to the subject include, e.g., the type or severity of the inflammatory bowel disease. For example, a subject having ulcerative colitis may require administration of a different dosage of a multi-epitope polypeptide, a mTOR metabolic inhibitor and/or an AMPK activator than a subject with Crohn's disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

The compositions are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, intradermally, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539. The composition can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. In some embodiments, the multi-epitope polypeptide and the mTOR metabolic inhibitor are therapeutically delivered to a subject by way of local administration. Effective doses for any of the administration methods described herein can be extrapolated from dose-response curves derived from in vitro or animal model test systems.

As used herein, the term subject means a mammalian subject. The term subject can be used interchangeably with the term patient. Exemplary subjects include, but are not limited to humans, monkeys, dogs, cats, mice, rats, cows, horses, camels, goats and sheep. In some embodiments, the subject is a human. In some embodiments, the subject has or is suspected of having an inflammatory bowel disorder, for example, Crohn's disease or ulcerative colitis. Optionally, the subject is diagnosed with an inflammatory bowel disease or at risk for developing an inflammatory bowel disease, for example. Crohn's disease or ulcerative colitis. The subject can be a human with an inflammatory bowel disease, wherein the subject has an increased anti-flagellin response, as compared to a control. The subject can be a human with an inflammatory bowel disease, wherein the subject has an increased anti-Lachnospiraceae flagellin response, as compared to a control. The subject can be a human with an inflammatory bowel disease, wherein the subject has an increased anti-Cbir 1 flagellin response, as compared to a control. Exemplary controls include, but are not limited to, a subject that is in remission, a healthy subject or a control value. In some methods, the subject can be a human subject that can be suspected of having an inflammatory bowel disease that can be treated with a polypeptide comprising one or more flagellin TCR epitopes and an mTOR metabolic inhibitor.

As used throughout, inflammatory bowel disease (IBD) is a group of intestinal disorders that cause chronic or prolonged inflammation of the digestive tract. Inflammation can occur anywhere along the digestive tract, for example, in the mouth, esophagus, stomach, small intestine and/or large intestine. Examples of inflammatory bowel disease include, but are not limited to, Crohn's disease and ulcerative colitis. In Crohn's disease, the condition most commonly affects the small intestine and colon, but it can occur anywhere in the gastrointestinal tract. Ulcerative colitis is typically limited to the colon, i.e., the large intestine.

As used herein, the terms, polypeptide, peptide, and protein are used interchangeably herein to refer to a polymer of amino acid residues. As used herein, the terms encompass amino acid chains of any length, including full-length proteins, wherein the amino acid residues are linked by covalent peptide bonds.

As used herein, a T cell receptor epitope is a peptide that can be recognized by T-cell receptors after a particular antigen has been intracellularly processed, bound to at least one MHC molecule and expressed on the surface of an antigen presenting cell as a MHC-peptide complex.

The term identity, as used in the context of polynucleotide or polypeptide sequences, refers to a sequence that has at least 80% sequence identity to a reference sequence. Alternatively, percent identity can be any integer from 80% to 100%. Exemplary embodiments include at least: 80%, 85%, 90%, 95%, or 99% identity, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The examples below are intended to further illustrate certain aspects of the methods and compositions described herein and are not intended to limit the scope of the claims.

### EXAMPLES

### Example I

Inflammatory bowel disease (IBD) is characterized as chronic inflammation in the gut mucosa, with Crohn's disease (CD) and ulcerative colitis (UC) as the two main forms. It is generally accepted that aberrant immune responses to environmental factors, especially to the gut microbiota, play a critical role in the pathogenesis of IBD. Although CD and UC share various genetic polymorphisms, dominant antigens identified in CD are considerably different from that in UC. Flagellins expressed by commensal Lachnospiraceae bacteria and CD4⁺ T cells specific to these flagellins are found in blood and intestinal lesions of Crohn's patients, suggesting that Lachnospiraceae flagellin is a keystone antigen driving gut inflammation in at least a subset of Crohn's patients. However, although Lachnospiraceae flagellin has been identified as an immunodominant antigen in CD at the protein level, the dominant B cell peptide epitopes and where they are located remain unclear.

The motility of various commensal and pathogenic bacteria, especially those that belong to the Proteobacteria and Firmicutes (reclassified as Bacillota now) phyla, is driven by their surface expression of flagella, which can consist of as many as 30,000 flagellin monomers. Although flagellin proteins have a vast span of diversity among different organisms, they share a characteristic molecular structure consisting of amino- and carboxyl-domains (D0 and D1) that are conserved within a given bacterial family. The amino- and carboxyl-domains are separated by a hypervariable region (D2 and D3 domains). In addition to conferring motility, flagellin is also one of the most potent immune activators known in the microbiota. As a result, flagellin proteins expressed by intestinal pathogens such as Salmonella spp. and Escherichia coli (E. coli) serve as major constituents mediating intestinal infection and inflammation. However, immune reactivity targeting flagellins expressed by commensal bacteria, especially those belong to the Lachnospiraceae family, is only elevated in patients with CD, but not in patients with UC or adult HC, despite the presence of abundant intestinal Lachnospiraceae bacteria in the latter.

Previously, using a microbiota protein antigen microarray, it was shown that ˜30% of CD patients in an regional IBD cohort recruited at the University of Alabama at Birmingham (UAB) had elevated serum IgG reactivity to more than 10 different flagellins expressed by Lachnospiraceae bacteria (from both human and mouse origin), and reactivity to individual flagellins was highly correlated to each other (Alexander, Katie L, et al “Human microbiota flagellins drive adaptive immune responses in Crohn's disease.” Gastroenterology 161.2 (2021): 522-535) suggesting that serum IgG response to Lachnospiraceae flagellins was driven by reactivity to shared epitopes, which remained unidentified. Consistently, these multi-flagellin reactive patients had significantly increased flagellin-specific effector/memory CD4⁺ T cells in the circulation, which could travel back to the gut and drive colitis. It was hypothesized that patients with multi-flagellin reactivity are more likely to develop disease complications in CD, and early identification of these patients through reactivity to the shared epitopes would greatly help with their disease treatment.

As described herein, for the first time, using a novel flagellin peptide microarray and a flagellin peptide cytometric bead array assay, a key IgG B cell epitope in Lachnospiraceae flagellins in CD patients, which locates at the “hinge region” between the D0 and D1 domain in the highly conserved amino-terminus (N-term) of Lachnospiraceae flagellins, was identified. Sero-reactivity to the dominant epitope was identified in regional adult CD patients, and in a multi-site-recruited cohort of pediatric CD patients. In addition, the magnitude of this epitope-specific reactivity was positively correlated with multi-flagellin reactivity in CD patients, and predicted the development of a complicated disease course in pediatric CD patients 2-3 years in advance. Interestingly, a robust level of serum IgG response to this dominant epitope was observed in over 90% of infants of diverse geographic origins, which peaked at one year of age, indicating that the elevated IgG reactivity to Lachnospiraceae flagellins in CD patients may result from an aberrant immune recall response of sensitization that occurred in early life.

### Study Design

The objective of this study was to identify immunodominant B cell peptide epitopes in the Lachnospiraceae bacterial family in patients with Crohn's disease and in healthy/non-IBD control infants, and correlate this immune reactivity with clinical disease course in the former. This was accomplished by performing a novel flagellin peptide array and flagellin peptide cytometric bead array with an adult discovery cohort containing patients with Crohn's disease, ulcerative colitis, and healthy controls, and a validation cohort of pediatric treatment-naïve Crohn's patients and non-IBD controls. Furthermore, a cohort of healthy and non-IBD control infants and their mothers from three distinct geographic locations was utilized for the discover of the homeostatic antibody response to the same dominant B cell epitope in Lachnospiraceae flagellins. Sample sizes were selected on the basis of expected variance and effect size in well-characterized experimental systems and were large enough to achieve a ≥90% power to detect differences >20% among groups. Replicated samples were randomly omitted. Experiments of the validation cohort were conducted blinded until data analyses,

### Patient Sample Collection

Peripheral blood samples along with medical metadata of IBD patients and healthy volunteers in the UAB cohort were collected at the Kirklin Clinic of UAB Hospital and in research labs in Birmingham, Alabama upon informed consent. All steps followed UAB's Institutional Review Board guidelines (IRB X300001155, X140515004, and F081003001) and the Declaration of Helsinki Principles. Serum samples of subjects in the RISK cohort were obtained from Dr. Subra Kugathasan at Emory University, USA Serum samples of subjects in the Uganda and US cohort were obtained from Dr. Edward N. Janoff at the University of Colorado Denver, USA. Serum samples of healthy and allergic subjects in the Sweden cohort were obtained from Dr. Maria C. Jenmalm at Linköping University, Sweden. Informed consent was obtained from both parents before inclusion.

### Motif Discovery

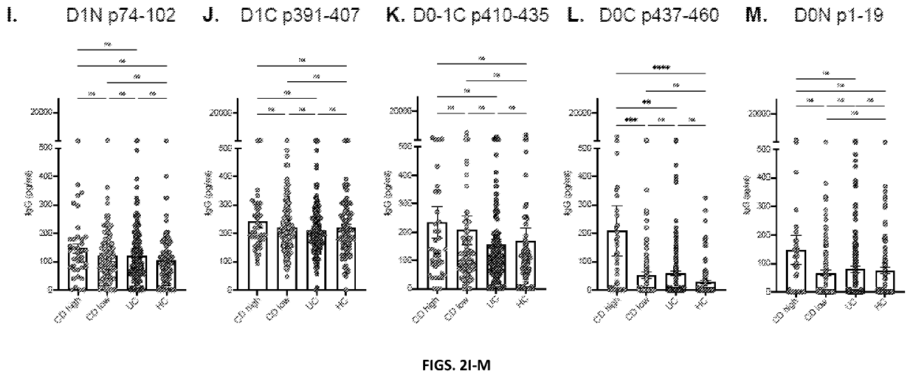

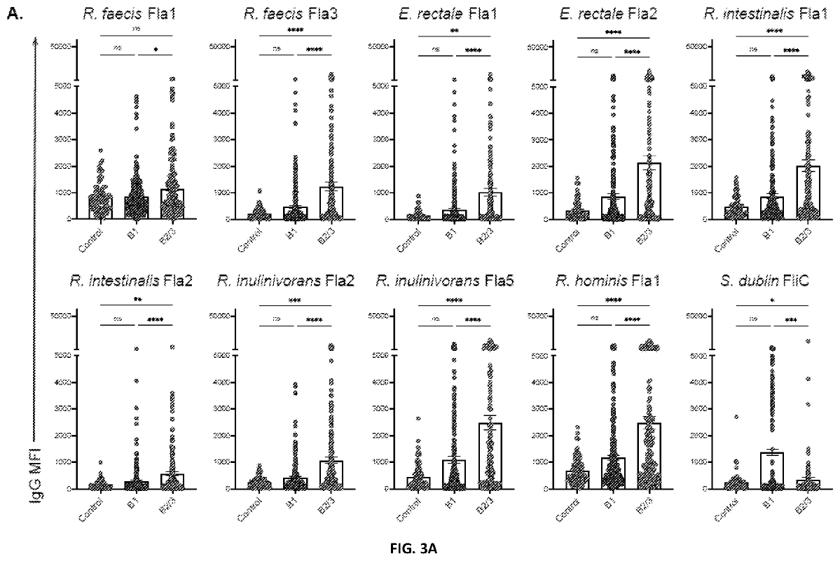

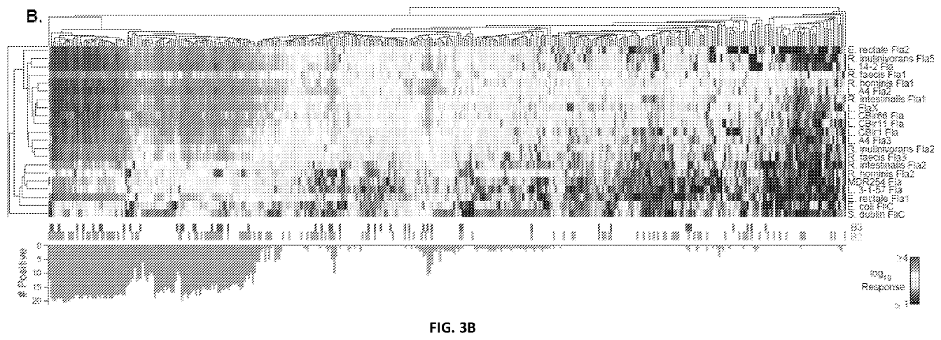

Protein sequences (D0-1N p0-75) of Lachnospiraceae flagellins included in the flagellin peptide microarray plus S. dublin FliC and E. coli FliC were analyzed with MEME Suite 5.5.0 for the discovery of recurring protein motifs with fixed length (6-35 width), with 0-order model of sequences used as the background model. Motifs discovered were ordered by E-value and the most significant motif was shown in FIG. 3.

### Microbiota Protein Microarray

Microbiota protein microarray was performed as previously described (Alexander et al.). In brief, recombinant bacterial flagellin proteins were expressed and diluted in 10 mM Tris (PH7.4) with 20% glycerol and 0.1% sodium dodecyl sulfate at 0.2 mg/ml. Antigens were printed in quadruplicate on slides coated with nitrocellulose pads (Maine Manufacturing, Samford, ME) using a Spotbot Personal microarrayer (ArrayIt, Sunnyvale, CA). Antigen printed pads were blocked with SuperBlock (ThermoFisher, Waltham, MA) for 1 hour before probed with serum samples (diluted at 1:100 in SuperBlock). After 1 hour, pads were washed 3 times with PBS plus 0.05% Tween 20, and then incubated with Dylight 550 Goat anti-human IgA (ImmunoReagents. Cat #GtxHu-001-E550NHSX, Raleigh, NC) and Dylight 650 Donkey anti-human IgG (Invitrogen, Cat #SA5-10129, Waltham, MA) at 1:1000 dilution for 1 hour. Finally, pads were washed 3 times with PBS plus Tween, air-dried, and scanned using GenePix 4000B imager (Axion, Atlanta, GA).

### Flagellin Peptide Microarray

1512 overlapping 15mer peptides derived from 19 Lachnospiraceae flagellins of human and mouse origin were synthesized and printed on individual glass slide as previously described (Legutki et al., “Scalable high-density peptide arrays for comprehensive health monitoring.” Nature communications 5.1 (2014). 4785). Slides were blocked with SuperBlock for 1 hour before they were probed with human sera (dilution factor indicated in corresponding Figure Legends). Slides were washed 3 times with PBS plus 0.05% Tween 20 after 1 hour incubation with serum samples, and then incubated with Dylight 550 Goat anti-human IgA and Dylight 650 Donkey anti-human IgG at 1:1000 dilution for 1 hour. Finally, slides were washed 3 times with PBS plus Tween, air-dried, and scanned using GenePix 4000B imager.

### Generation of Flagellin Peptide Microarray Heatmaps

UAB cohort: 1477 of the original 1512 peptides are depicted (columns in the heatmap). A peptide was excluded if more than 190 subjects had signal greater than 2.16 (i.e. log 10(signal+1)=0.5). 265 of the original 288 subjects are depicted (rows in the heatmap). A subject was excluded if more than 500 peptides had signal greater than 2.16. These thresholds were determined by identifying cutoffs in long tails of distributions subjects and peptides. RISK cohort: 474 peptides and 120 subjects are depicted.

### Flagellin Peptide Cytometric Bead Array

8 conserved peptides from Lachnospiraceae flagellins were synthesized, with biotin and a GSGSG linker attached to their N-terminus. DIN p41-59 and D0-1N p25-59 include a 50/50 mixture of 2 consensus peptides with one amino acid different, respectively. Sequence of each peptide is listed as follows:

D0N p1-19:
(SEQ ID NO: 19)
[Biotin]GSGSGMVVQHNLRAMNSNRMLGIT[COOH]
D0N p25-44:
(SEQ ID NO: 20)
[Biotin]GSGSGKSTEKLSSGYKINRAADDAA[COOH]
D1N p41-59:
(SEQ ID NO: 21)
[Biotin]GSGSGDDAAGLTISEKMR(S/K)QIRGL[COOH]
D0-IN p25-59:
(SEQ ID NO: 22)
[Biotin]GSGSGKSTEKLSSGYKINRAADDAAGLTISEKM
R(S/K)QIRGL[COOH]
D1N p74-102:
(SEQ ID NO: 32)
[Biotin]GSGSGQTAEGALTEVHDMLQRMNELAVQAAN
GTN[COOH]
D1C p391-407:
(SEQ ID NO: 33)
[Biotin]GSGSGLGAVQNRLEHTINNLDN[COOH]
D0-1C p410-435:
(SEQ ID NO: 34)
[Biotin]GSGSGENTTAAESQIRDTDMATEMVKYSNN
N[COOH]
D0C p437-460:
(SEQ ID NO: 35)
[Biotin]GSGSGLAQAGQSMLAQSNQANQGVLSLL
G[COOH]
Note that () indicates that it's a 50/50 mix
of peptides at this position.

Flagellin peptide cytometric bead array was performed as previously described (Rosenberg, et al. “A high-throughput multiplex array for antigen-specific serology with automated analysis.” bioRxiv (2023): 2023-03). In brief, biotinylated flagellin peptides were conjugated on to Streptavidin coated 4 μm polystyrene beads (Spherotech, Inc. Cat #PAK-4067-8K, Lake Forest, IL). Beads conjugated with each individual peptide have different fluorescent intensity in the APC/Cy7 channel so that they can be differentiated from each other by flow cytometry. After incubation of 40 μl diluted serum samples (serum samples were diluted at 1:200 and 1:600 in SuperBlock to ensure sufficient detection of epitope-specific IgG response; IgM and IgA responses were detected at 1:200 and 1:600 with IgG, respectively) from individuals with CD, UC, and HCs with the array of peptide-bound beads, antibody response to individual peptides was detected with different fluorescently labeled secondary antibodies (Goat anti-human IgM-AF488, Southern Biotech, Cat #2020-30, Birmingham, AL; Goat anti-human. IgG-PE, SouthernBiotech. Cat #2040-09; Goat anti-human IgA-PE, SouthernBiotech, Cat #2050-09; and Goat anti-human IgG-AF488, SouthernBiotech, Cat #2040-30) using CytoFLEX cytometer (The Beckman Coulter Life Sciences. Indianapolis, IN).

### Calculation of Epitope-Specific Antibody Concentrations in Flagellin Peptide Cytometric Bead Array

Biotinylated Goat F(ab)2 anti-human isotype antibodies (anti-human IgM, SouthernBiotech, Cat #2022-01; anti-human IgA. SouthernBiotech, Cat #2052-01, and anti-human IgG, SouthernBiotech, Cat #2042-01) were conjugated on to selected peaks of Streptavidin coated 4 μm polystyrene capture beads, respectively, and incubated with 0.75× serial dilutions of purified polyclonal human antibodies (IgG, SouthernBiotech. Cat #0150-01; IgM, SouthernBiotech, Cat #0158L-01; IgA, SouthernBiotech, Cat #0155K-01) at known concentrations. Standard curves were obtained based on MFI detected by secondary antibodies bound using CytoFLEX cytometer. Flow cytometry standard (FCS) files of both samples and the standard curve were applied to a set of MATLAB programs (https://github.com/UAB-Immunology-Institute/cba-toolbox) for the quantification of peptide-specific antibodies in the corresponding isotype(Rosenberg, et al.).

### Statistical Analysis

Statistical analyses were performed with GraphPad PRISM, version 9.5, with nonparametric Kruskal-Wallis test or two-way ANOVA as indicated. Dunn's or Tukey's multiple comparisons test was used when comparing multiple groups. All statistical tests were 2-sided. Specific tests used and values of n are stated in corresponding figure legends. Data were presented as means±SEM. The differences were considered statistically significant at P<0.05 (*P<0.05, **P<0.01, ***P<0.001, ****P<0.0001) and ns indicates not significant.

### Results

### Cohort Description

Three cohorts were used in this study, including a discovery cohort of adult IBD patients and HC recruited at UAB (Alexander, et al.), a validation cohort of treatment-naïve pediatric CD patients and non-IBD controls collected at 28 sites in the USA and Canada (the RISK Stratification Study)(Kugathasan, et al. “Prediction of complicated disease course for children newly diagnosed with Crohn's disease: a multicentre inception cohort study.” The Lancet 389.10080 (2017): 1710-1718), and a healthy and non-IBD control cohort of infants and their mothers recruited in Uganda, the USA (Gaensbauer, et al. “Impaired Haemophilus influenzae type b transplacental antibody transmission and declining antibody avidity through the first year of life represent potential vulnerabilities for HIV-exposed but-uninfected infants.” Clinical and Vaccine Immunology 21.12 (2014): 1661-1667), and Sweden (Christmann, et al. “Human seroreactivity to gut microbiota antigens.” Journal of Allergy and Clinical Immunology 136.5 (2015): 1378-1386).

### Lachnospiraceae Flagellin Peptide Microarray Identifies Key IgG Binding Epitopes in CD Patients

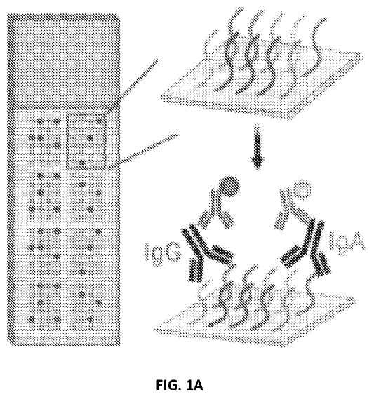

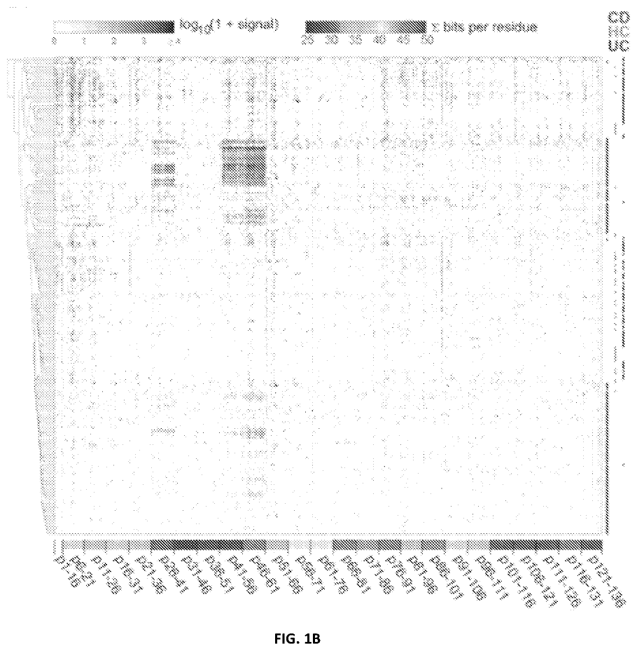

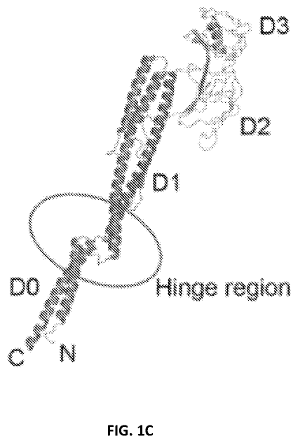

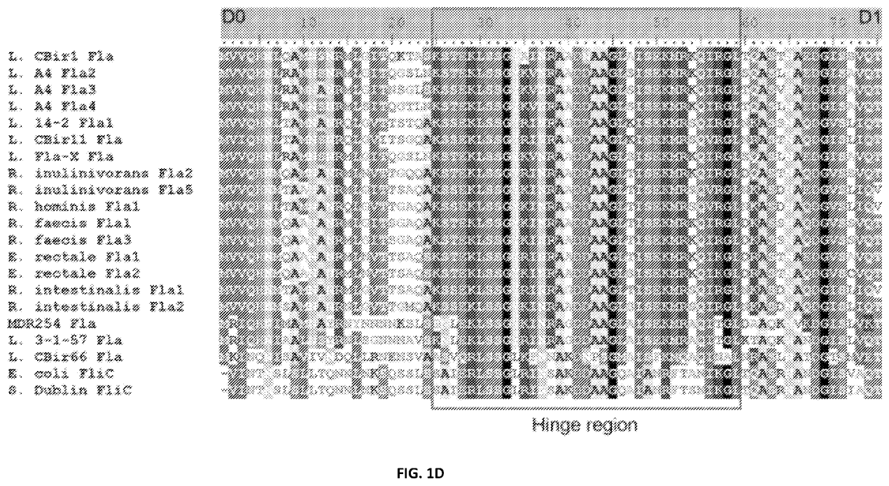

With a previous microbiota protein microarray, Pearson correlation analysis showed that serum IgG response to most Lachnospiraceae flagellins strongly correlated with each other in CD patients (Alexander, et al.). This observation suggested that serum IgG reactivity in CD patients was targeting shared epitopes among different Lachnospiraceae flagellins. To test this hypothesis, a novel flagellin peptide microarray consisting of 1512 peptides derived from 19 different Lachnospiraceae flagellins of both human and mouse origin, was constructed. These were sequential 15mer peptides overlapping by 5 amino acids, which covered the entire sequences of 19 flagellin proteins. Sera from CD patients, HC, and UC patients were probed against the array, and peptide-specific IgG or IgA reactivities were detected with fluorescently labeled secondary antibodies (FIG. 1A). In the heatmap where serum IgG reactivity to peptides in the D0 and D1 domains of the N-term of Lachnospiraceae flagellins is shown (FIG. 1B), those in CD patients clustered apart from those in UC patients and HC, whereas the response to Lachnospiraceae flagellin peptides in UC and HC subjects was not different. Notably, reactivities from CD sera fell in two major clusters, with increased IgG binding identified at p26-41 and p41-61 between the D0 and D1 domains in the amino chain of Lachnospiraceae flagellins (FIGS. 1B and 1C), which was not seen in UC or HC subjects. Thus, binding to these conserved epitopes was specific for Crohn's disease. There was no significant difference seen in serum IgG reactivity among CD, UC, and HC subjects against other peptides included in the microarray. Differences in the IgA response to Lachnospiraceae flagellin peptides among groups were not observed.

Sequence analysis and motif discovery analysis revealed that, regardless of human or mouse origin, the majority of the 19 Lachnospiraceae flagellins included in the peptide microarray (aligned in FIG. 1D) have significant amino acid homology in both the N-term and C-term of their D0 and D1 domains. Specifically, such homology peaks at the “hinge region” between the D0 and D1 domains at the N-terminus (FIGS. 1D and 1E), which is significantly different from flagellins expressed by Proteobacteria Salmonella dublin (S. dublin) and E. coli, although the latter two are homologous to each other. As shown in FIG. 1D, the hinge region lies between the D0 and the D1 domains of the flagellins and is highly conserved in Lachnospiraceae but not Proteobacteria Salmonella Dublin FliC or E Coli FliC, which have a very different hinge region. Multiple, likely B cell epitopes were predicted in this hinge region when Bepipred Linear Epitope Prediction 2.0 was performed through Immune Epitope Database, consistent with this region being highly immunogenic. The dominant B cell epitope in Lachnospiraceae flagellins identified in CD patients with the flagellin peptide microarray locates right at this “hinge region”. It includes sub-epitopes p25-44, which locates at the D0 domain, and p41-59 which locates at the D1 domain. The dominant Lachnospiraceae flagellin B cell epitope in CD, D0-1N p25-59, is referred to herein as the “hinge peptide” (FIG. 1E).

### Serum IgG Multi-Flagellin Reactivity in CD Patients is Driven by Reactivity to the Dominant Lachnospiraceae Flagellin Peptide Epitope

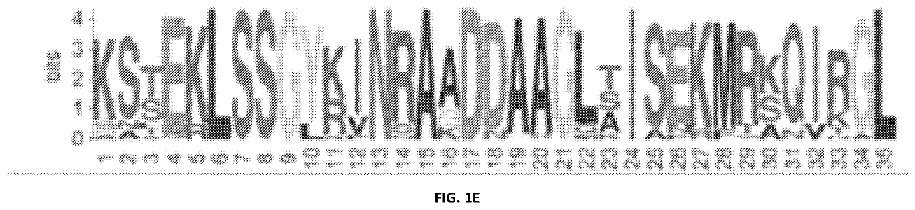

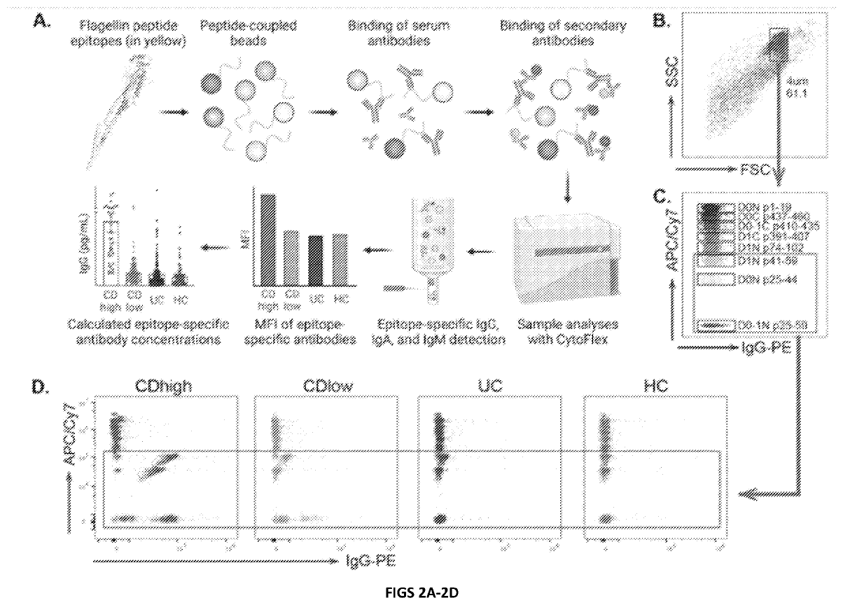

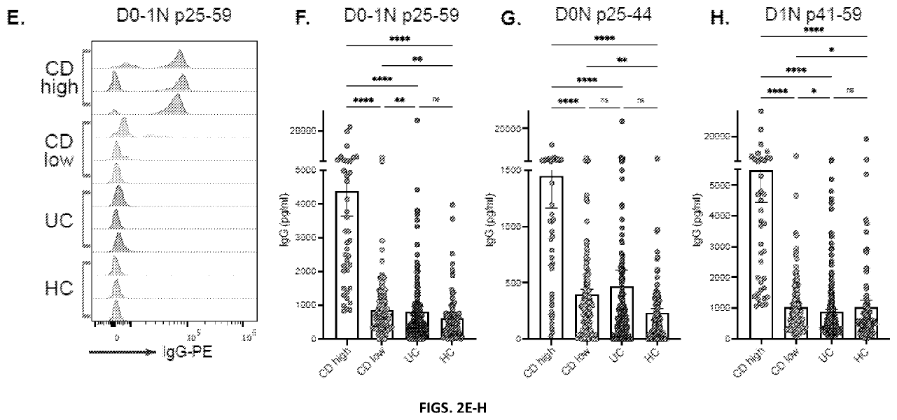

In order to confirm the findings of the flagellin peptide microarray, a flagellin peptide cytometric bead array, where serum IgG, IgA, and IgM reactivity to different bead-bound antigens can be simultaneously detected in a multiplexed fashion (FIG. 2A) was developed. Eight biotinylated conserved peptides (DON p1-19, DON p25-44, DIN p41-59, D0-1N p25-59, D1N p74-102, D1C p391-407, D0-1C p410-435, DOC p437-460) covering the N-term (amino domain) and C-term (carboxy domain) of D0 and D1 domains of Lachnospiraceae flagellins were synthesized (Table 1) and individually conjugated to 4-micron fluorescent beads (FIGS. 2B and 2C).

TABLE 1
Flagellin Peptide Array

| | | SEQ |
|---|---|---|
| Peptide | | ID |
| Position | Peptide Sequence | NO |
| Domain | MVVQHNLRAMNSNRMLGIT[COOH] | 2 |
| D0 amino | | |
| end | | |
| p1-19 | | |
| Domain | KSTEKLSSGYKINRAADDAA[COOH] | 3 |
| D0 amino | | |
| end | | |
| p25-44 | | |
| Domain | DDAAGLTISEKMR(S/K)QIRGL[COOH] | 4 |
| D1 amino | | |
| end | | |
| p41-59 | | |
| Domain | KSTEKLSSGYKINRAADDAAGLTISEK | 5 |
| D0-D1 | MR(S/K)QIRGL[COOH]* | |
| amino end | | |
| p25-59 | | |
| Domain | QTAEGALTEVHDMLQRMNELAVQAA | 6 |
| D1 amino | NGTN[COOH] | |
| end | | |
| p74-102 | | |
| Domain | LGAVQNRLEHTINNLDN[COOH] | 7 |
| D1 | | |
| carboxy | | |
| end | | |
| p391-407 | | |
| Domain | ENTTAAESQIRDTDMATEMVKYSN | 8 |
| D1-D0 | NN[COOH] | |
| carboxy | | |
| end | | |
| p410-435 | | |
| Domain | LAQAGQSMLAQSNQANQGVLSLLG[COOH] | 9 |
| D0 | | |
| carboxy | | |
| end | | |
| p437-460 | | |

Each peptide was linked to a biotin moiety by a GSGSG (SEQ ID NO: 1) linker as set forth below:

D0N p1-19:
(SEQ ID NO: 19)
[Biotin]GSGSGMVVQHNLRAMNSNRMLGIT[COOH]
D0N p25-44:
(SEQ ID NO: 20)
[Biotin]GSGSGKSTEKLSSGYKINRAADDAA[COOH]
D1N p41-59:
(SEQ ID NO: 21)
[Biotin]GSGSGDDAAGLTISEKMR(S/K)QIRGL[COOH]
D0-1N p25-59:
(SEQ ID NO: 22)
[Biotin]GSGSGKSTEKLSSGYKINRAADDAAGLTISEKM
R(S/K)QIRGL[COOH]
D1N p74-102:
(SEQ ID NO: 32)
[Biotin]GSGSGQTAEGALTEVHDMLQRMNELAVQAANGT
N[COOH]
D1C p391-407:
(SEQ ID NO: 33)
[Biotin]GSGSGLGAVQNRLEHTINNLDN[COOH]
D0-1C p410-435:
(SEQ ID NO: 34)
[Biotin]GSGSGENTTAAESQIRDTDMATEMVKYSNN
N[COOH]
D0C p437-460:
(SEQ ID NO: 35)
[Biotin]GSGSGLAQAGQSMLAQSNQANQGVLSLL
G[COOH]
Note that () indicates that it's a 50/50 mix
of peptides at this position.

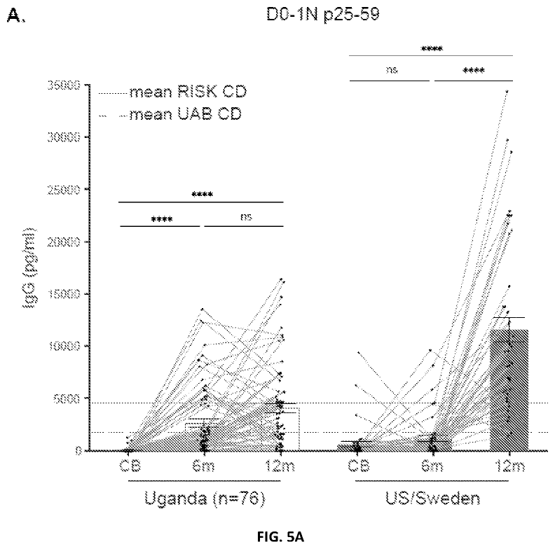

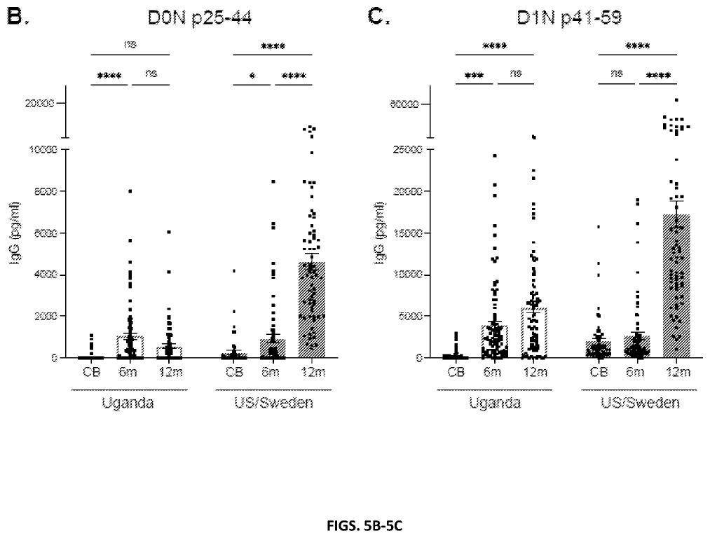

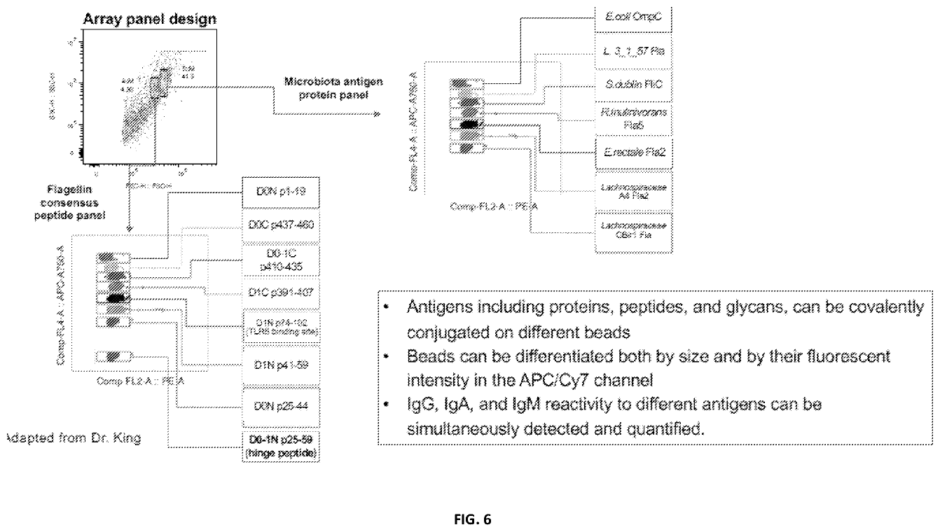

For certain epitope sequences a mixture of two different peptides were included such that half had a given residue (e.g., serine) at a specific position whereas others had the alternative amino acid (e.g., lysine) at the specific position. Different peptides were attached to cytometric beads that incorporate different amounts or types of fluorophores and/or are of different size (4 mm or 5 mm). The beads were distinguished from one another due to the incorporated fluorophore, shown along the Y axis in FIG. 6. IgG binding to the beads was detected and the mean fluorescent intensity (MFI) was quantitated by anti-IgG, anti-IgA, or anti-IgM labelled with a different fluorophore, shown along the X axis. The assay was multiplex since all the beads were used together and the flow cytometer can separate the beads by size and by incorporated fluorophore using the APC/Cy7 channel.

Beads conjugated with different peptides can be differentiated by their fluorescent intensity in the APC/Cy7 channel with flow cytometry (FIG. 2C). Serum samples (UAB cohort) from CD patients with multi-flagellin reactivity on the microbiota protein microarray (≥10 different flagellins, CDhigh), CD patients who reacted to less than 10 or no Lachnospiraceae flagellins (CDlow), UC patients and HC were assayed and IgG, IgA, and IgM reactivity to individual peptides were detected with fluorescently labeled secondary antibodies. Standard curves using human IgA, IgM, and IgG capture beads and known concentrations of polyclonal human IgA, IgM, and IgG were obtained and applied to quantify peptide-specific antibodies, allowing cross-comparison of samples run at different times. Data from this cytometric bead array assay strongly validated the results we obtained from the flagellin peptide microarray (FIG. 2D). IgG reactivity to the “hinge peptide” (D0-1N p25-59) is highly represented in CDhigh patients compared to CDlow, UC, and HC (FIG. 2D-F). In addition, CDhigh patients have significantly elevated serum IgG specific to the sub-epitopes (DON p25-44 and DIN p41-59) compared to CDlow patients, UC patients, and HC (FIGS. 2G and 2H), but not to other conserved peptides of the Lachnospiraceae flagellins including DON p1-19, D1N p74-102, D1C p391-407, and D0-1C p410-435 (FIGS. 2I-K, and 2M). These data indicate that multi-flagellin IgG reactivity in Crohn's patients is driven by reactivity to the hinge region of the N-terminus. Of note, CDhigh patients also have significantly elevated serum IgG reactivity to DOC p437-460 (FIG. 2L), which is the terminal conserved region of Lachnospiraceae flagellin C-terminus, although the magnitude of reactivity is much lower than that to the N-term hinge peptide. Serum IgA and IgM responses to individual conserved peptides were also analyzed, but consistent with the microbiota protein microarray and flagellin peptide microarray data, no differential reactivity among groups was observed.

Correlation of Cytometric Flagellin Epitope Bead Array with Flagellin Protein Microarray

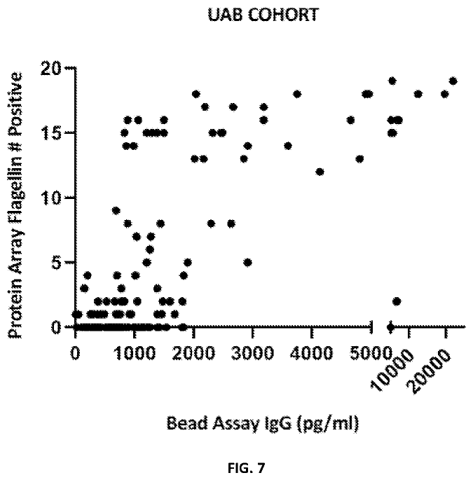

UAB cohort sera from adult IBD patients being treated in the UAB IBD Center, 70% of which have had surgery and are under treatment with various biologics, were used in two binding assays. Binding of IgG to hinge peptide beads and the number of flagellins bound by sera on the flagellin protein microarray were assessed. Quantification was performed by interpolating the MFI of samples against a standard curve constructed of anti-IgG binding to human IgG coated beads. FIG. 7 shows strong correlation between the number of IgG anti-flagellin positive sera on the flagellin protein microarray on the Y axis and the IgG anti-hinge peptide in pg/ml on the X axis.

### Lachnospiraceae Hinge Peptide is the Dominant B Cell Epitope in Pediatric CD Patients and Predicts the Development of Disease Complications

Although it was possible to confidently identify the dominant B cell peptide epitope in CD patients using the IBD cohort recruited at UAB, there were several limitations involved. First, it is a regional adult cohort of IBD, which might not represent a broader IBD population. Also, the refractory nature of this cohort (rate of biologic therapy in CD was 78% and rate of surgery in CD was 66%) limited the power to analyze the association between the epitope-specific IgG sero-response and disease complications in CD. Therefore, it was decided to validate this finding using a multi-site-recruited pediatric cohort consisting of patients who were newly diagnosed with CD as well as non-IBD controls (the RISK Stratification Study). CD patients in this cohort represented the variety of disease phenotypes regarding disease location, severity, and behavior, and they were recruited at diagnosis prior to any treatment. Furthermore, patients were followed up to 36 months after the initial sample collection, and their disease course was well-documented, thus the prospective nature of this cohort enabled us to test whether serum IgG to the dominant epitope in CD patients was able to predict the development of disease complications later. In specific, the Montreal classification was used and developing stricturing (B2) or penetrating (B3) disease behavior was defined as a complicated disease course. To confirm that pediatric CD patients in the RISK cohort are reactive to Lachnospiraceae flagellins, the microbiota protein microarray was perfomed as previously described (Alexander, et al.) Sera at recruitment of CD patients who remained inflammatory (B1 behavior) at 3-year follow-up, CD patients who developed strictures or penetrating disease (B2/3 behavior) at 3-year follow-up, and non-IBD controls were probed with 19 Lachnospiraceae flagellins of human and mouse origin, in addition to S. dublin FliC and E. coli FliC.

A previous study using this cohort has reported an augmented serum IgG specific to L. CBir1 Fla in 35% of CD patients who remained B1 at follow-up, and in 62% of CD patients who developed B2/3 at follow-up. Consistent with this data, results provided herein showed that CD patients that remained B1 exhibited a trend of increased, although not significant, serum IgG reactivity to Lachnospiraceae flagellins when compared to control subjects (FIG. 3A). In contrast, CD patients that developed B2/3 at follow-up showed significantly elevated serum IgG reactivity to the majority of Lachnospiraceae flagellins compared to controls and to patients who remained B1 (FIG. 3A). Consistent with what was observed in the adult CD cohort recruited at UAB, a subset of pediatric CD patients also exhibited serum IgG reactivity to the majority of Lachnospiraceae flagellins tested (FIG. 3B), and the proportion of patients that were multi-flagellin reactive was significantly higher in those who developed B2/3 at follow-up (FIGS. 3C and 3D).

Next, whether the Lachnospiraceae flagellin hinge peptide also served as the dominant B cell epitope in CD patients in the RISK cohort was tested by performing the flagellin peptide microarray with sera of the multi-flagellin reactive CD patients. Similar to what was observed in the adult IBD cohort recruited at UAB, serum IgG response in multi-flagellin reactive CD patients in the RISK cohort mainly targeted p26-41 and p41-61 between the D0 and D1 domains in the N-term of Lachnospiraceae flagellins. Finally, patient and control sera from the whole cohort were assayed with the flagellin peptide cytometric bead array. Similar to adult CD patients, the dominant serum IgG binding sites in pediatric CD patients located at the conserved Lachnospiraceae flagellin hinge region between D0 and D1 domains in the N-term (D0-1N p25-59, DON p25-44, and DIN p41-59, FIGS. 3E-G). IgG sero-reactivity to other peptides was either not significantly different among groups (DIN p74-102 and D1C p391-407) or at a much lesser magnitude compared to the hinge peptide (D0-1C p410-435, DOC p437-460, and D0N p1-19) Moreover, response to the N-term hinge peptide in patients who developed B2/3 at follow-up was significantly higher than that of patients who remained B1 at follow-up, indicating the prognostic value of hinge peptide specific serum IgG in CD patients early on, even at initial disease diagnosis. In conclusion, the dominant B cell peptide epitope in Lachnospiraceae flagellins in CD patients were validated with both the flagellin peptide microarray and flagellin peptide cytometric bead array, using the RISK cohort which represents a broad variety of disease phenotypes and behaviors. More importantly, results from the flagellin peptide cytometric bead array demonstrated that elevated serum IgG to Lachnospiraceae flagellin hinge peptide was associated with stricturing or penetrating disease at 3-year follow-up, thus can be used to predict the development of disease complications in CD.

### Differentiating Reactivity to the Hinge Region by IgG, IgA, and IgM

Serum samples from Crohn's patients with anti-flagellin reactivity to more than 10 different flagellins (CD high), Crohn's patients with anti-flagellin reactivity to less than 10 different flagellins or have no increased anti-flagellin reactivity (CD low), patients with ulcerative colitis (UC) (typically lacking elevated anti-flagellin reactivity), and healthy controls were assayed for IgG. IgA, and IgM reactivity to the hinge region. As shown in FIG. 8, IgG reactivity to the hinge region was much higher in CD high patients, compared to CD low patients, UC patients, and healthy controls, but IgA and IgM antibodies from the same serum samples did not have increased binding to the hinge region.

Serum IgG to Lachnospiraceae Hinge Peptide Strongly Correlates with Multi-Flagellin Reactivity in CD

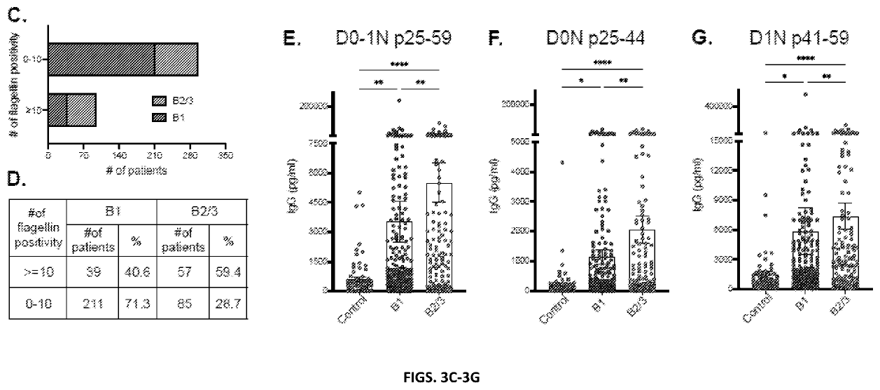

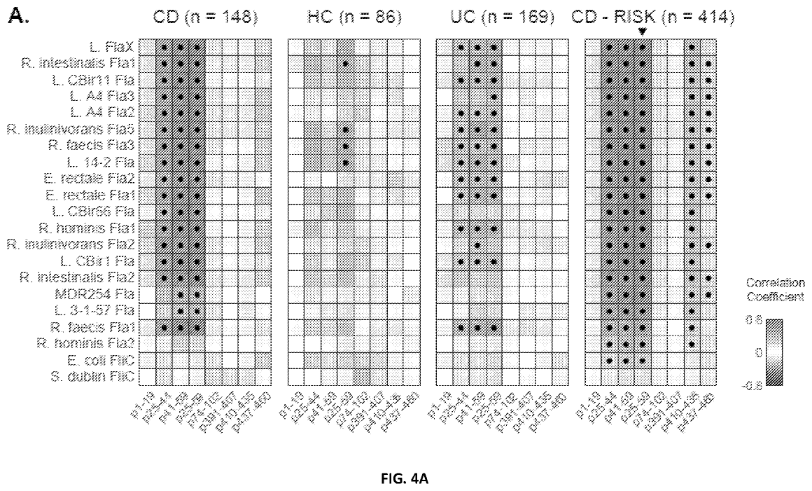

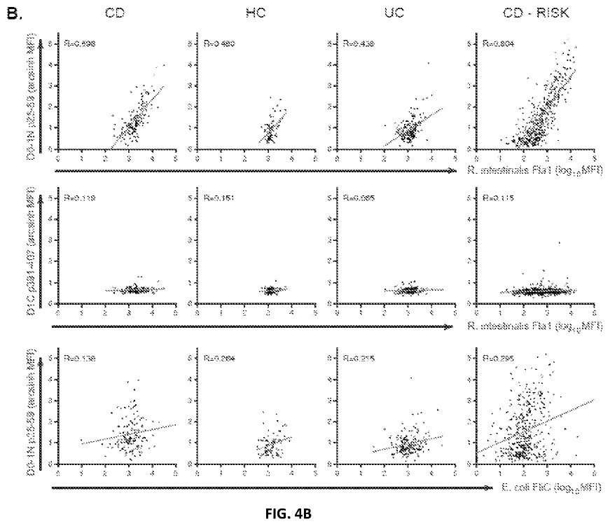

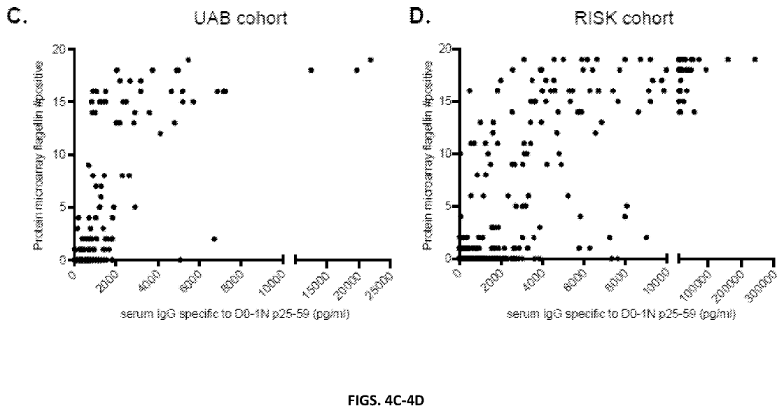

Next, whether serum IgG reactivity to the conserved peptides included in the flagellin peptide cytometric bead array correlates with reactivities to Lachnospiraceae flagellins included in the microbiota protein microarray was determined. First, Pearson correlation analysis was performed between serum IgG reactivity against individual flagellins and serum IgG reactivity specific to individual peptide epitopes for CD, HC, and UC subjects from the UAB cohort and CD patients from the RISK cohort (FIG. 4A). The most significant correlations with serum reactivities specific to Lachnospiraceae flagellin proteins were observed in serum reactivities against D0N p25-44, DIN p41-59, and D0-1N p25-59, i.e. the hinge peptide, especially in CD patients from the UAB cohort and the RISK cohort. Sero-reactivities to the hinge peptide did not universally correlate with all flagellins tested, in that the correlation with that against Proteobacteria flagellins such as E. coli FliC and S. dublin FliC was significantly reduced. Moreover, sero-reactivity to the C-term conserved epitopes D0-1C p410-435 and DOC p437-460 showed slight significance in correlating with that to Lachnospiraceae flagellin proteins in RISK cohort CD patients. To better view the correlation in each subject, we plotted individual sero-reactivity against the most significant peptide, D0-1N p25-59, and one of the least significant peptides, D1C p391-407, with that of R. intestinalis Fla1, and E. coli FliC, respectively (FIG. 4B). Reactivity to D0-1N p25-59, especially in CD patients from both UAB and the RISK cohort correlated significantly with that of R. intestinalis Fla1, which was expressed by bacteria in the Lachnospiraceae family, but poorly with E. coli FliC. In contrast, reactivity to D1C p391-407 did not correlate with that of R. intestinalis Fla1 in any subject group, demonstrating that reactivity to the hinge peptide, but not to other conserved flagellin peptides, underlies sero-reactivity to Lachnospiraceae flagellins. Furthermore, a good concordance was found between sero-reactivity to the hinge peptide obtained from the flagellin peptide cytometric bead array and flagellin sero-positivity determined by the microbiota protein microarray in both the UAB cohort and the RISK cohort (FIGS. 4C and 4D). This further demonstrates that multi-flagellin reactivity in CD patients is largely due to IgG binding to the dominant hinge peptide epitope.

### Serum IgG Reactivity to Lachnospiraceae Flagellin Hinge Peptide is a Homeostatic Response in Most Infants

A previous study showed a surprisingly similar level of serum IgG reactivity to Firmicutes flagellins in healthy and allergic Swedish infants at 2 years of age compared to that in adult CD patients (Christmann, et al.). Whether this high level of flagellin reactivity existed universally in infants raised in distinct environments was investigated. To test this hypothesis, sera were obtained from HIV-exposed but uninfected healthy infants from Uganda, healthy and allergic infants from Sweden, and healthy infants from the USA at 6 and 12 months of age, as well as their cord blood representing antibody reactivity of the mothers. Data from the Swedish and the American infants were grouped together, representing reactivity from developed countries, whereas data from the Ugandan infants represented reactivity in developing countries.

Serum samples were probed with the flagellin peptide cytometric bead array and the level of IgG reactivity to the hinge peptide in each individual at different time points was linked and compared to that of CD patients in both the UAB cohort and RISK cohort. Hinge peptide specific IgG response in the cord blood of both Uganda and US/Sweden cohorts was low and comparable to that identified in adult HCs from the UAB cohort (FIG. 5A). Consistent with our previous microbiota protein microarray data(30), serum IgG response to the hinge peptide was significantly elevated at 12 months of age in both cohorts (FIG. 5A), indicating that IgG sero-reactivity to Lachnospiraceae flagellins is a homeostatic response present in infants regardless of environmental exposures, and this heightened response is driven by reactivity to the hinge epitope. Accompanied with this, there was an increase in sero-reactivity to other microbiota antigens at 12 months of age (Christmann, et al.), reflecting the development of homeostatic IgG response to endogenous gut microbiota in early life. Interestingly, increase in hinge-specific sero-reactivity already happened at 6 months of age in Ugandan infants, but remained low in infants in the US/Sweden cohort; whereas reactivity was much greater in US/Sweden infants than Ugandan infants at 12 months of age (FIG. 5A). Furthermore, sero-reactivity to the hinge peptide was attributed to both sub-epitopes (D0N p25-44 and D1N p41-59) in the US/Sweden cohort (FIGS. 5B and 5C), but there was a lack of potent sero-reactivity to sub-epitope DON p25-44 in Uganda infants at 12 months of age (FIG. 5B). Distinct from response to the hinge peptide, serum IgG reactivity to the other peptide epitopes tested in the cytometric bead array in Ugandan infants exhibited a significant increase at 6 months of age and started to decline at 12 months of age, which was not observed in the US/Sweden cohort (FIGS. 5A-C). Collectively, these data indicate that there is an environmental contribution to the kinetics of the development of homeostatic IgG response to the gut microbiota.

The dynamic interactions of the host with its microbiota begins at birth and continues throughout the life of the host. The composition of human gut microbiota changes over a lifetime, but most critically early in life where the microbiota is unstable until 3 years of age. A collection of genes is important in establishing and maintaining homeostasis with the microbiota, and when such genes are deleted from mice, colitis ensues. A similar process occurs in humans resulting in IBD, which eventuates in immune reactivity to the microbiota. Data provided herein provides further insight into such response, identifying a shared dominant B cell peptide epitope in CD patients for the first time. Importantly, heightened sero-reactivity to this dominant epitope at CD diagnosis is able to predict the future development of disease complications up to 3 years, making the Lachnospiraceae flagellin hinge peptide a promising biomarker candidate for both CD diagnosis, prognosis, and treatment.

Another important finding was that most human infants at one year of age have a strong IgG response to Lachnospiraceae flagellin, targeting the exact dominant peptide epitope identified in CD patients. CD4+ T cell help is required for B cell responses to peptides, and T cells are restricted by major histocompatibility complex (MHC) class II molecules. The almost universal IgG response to the hinge peptide despite human immune diversity indicates that the T cell response is not restricted to any MHC class II allele. Indeed, T cell epitopes that are promiscuous, i.e., can bind to multiple MHC class II alleles, are enriched in Lachnospiraceae flagellins. Starting from 6 months, infants are often first introduced to solid food and thus experiencing a massive shift in the composition of the gut microbiota. This is also a critical time window for the colonization and diversification of Firmicutes in the human gut. The increased serum IgG response to Lachnospiraceae flagellins reflects an augmented homeostatic T and B cell response to the colonizing bacteria, in that this process does not involve intestinal inflammation. Interestingly, the magnitude of hinge-specific serum IgG is higher in Sweden/US infants than that of Ugandan infants. Interestingly, this heightened sero-reactivity to Lachnospiraceae flagellins declines with age, in that the flagellin epitope response is hardly detected in healthy adults, consistent with an active regulation of this B cell reactivity and the associated T cell reactivity. It is unclear which immune pathways participate in the downregulation of such immune responses, and the robust IgG sero-reactivity to Lachnospiraceae flagellins in CD patients indicates a loss of immunological control. Because flagellins are potent activators of several innate immune receptors, it is possible that defective innate immune pathways are involved. Interestingly, sero-reactivity to the Lachnospiraceae flagellin hinge peptide was also found elevated in a subset of patients with severe chronic fatigue syndrome (Vogl, et al. Sci Adv 8, eabg2422, (2022)). These data are consistent with a homeostatic immune response to flagellin in infancy being converted to a pathologic response later in life.

### Example II

### Antibody Production

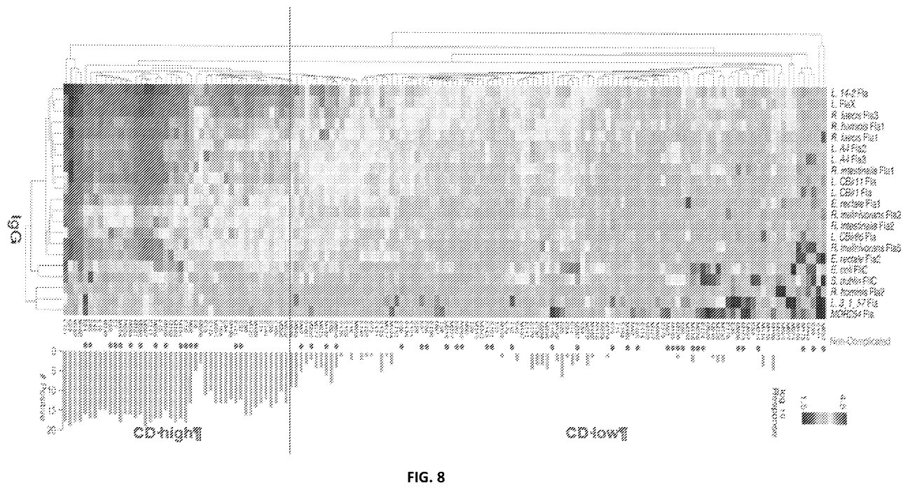

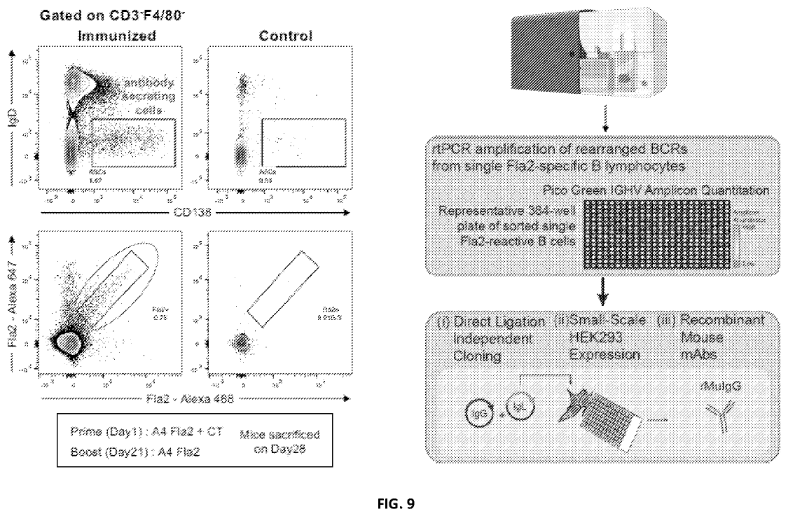

Mice were immunized with A4 Flagellin 2 with cholera toxin (CT) as an adjuvant (Prime on Day 1) and were boosted on Day 21 with A4 Flagellin 2. Animals were sacrificed on Day 28, and antigen producing B cells were identified as IgD-CD138+ cells. B cells producing anti flagellin antibodies were further identified by binding both A4 Fla2—Alexa647 and A4 Fla2—Alexa 488 flourophores and were sorted as single B cells into a 384 well plate. The V region binding site was sequenced, and cloned into plasmids, which were then expressed in HEK293 cells. FIG. 9 shows the workflow for rapid recombinant monoclonal antibody generation using IgClic technology.

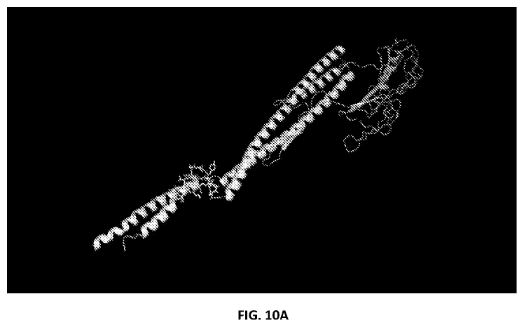

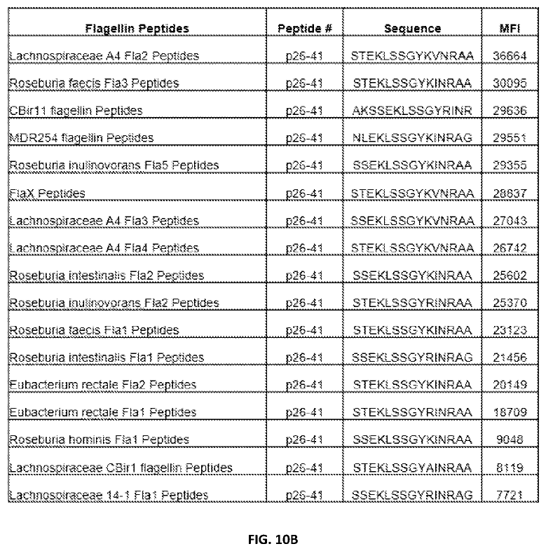

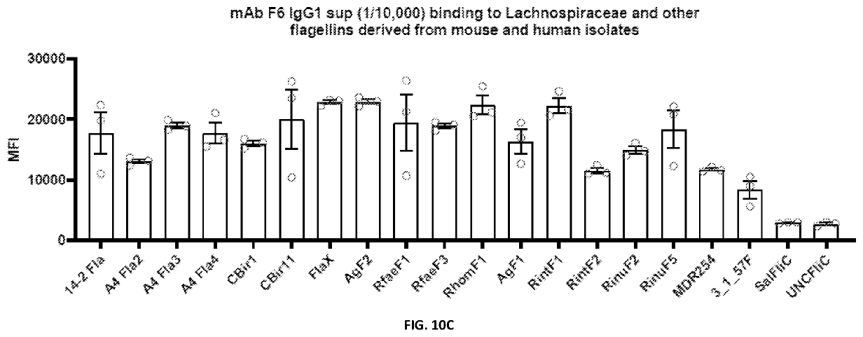

The Fla2 immunized B cells generated around 38 monoclonal antibodies from the first 96 single B cells tested. One of these, designated monoclonal antibody F6 (mAb F6), bound to the 25-44 epitope of the amino domain hinge region. The predicted 3-dimensional structure of Fla2 is shown in FIG. 10A. mAbF6 binds almost all Lachnospiraceae flagellins, but not to Salmonella FliC or E. coli FliC. FIG. 10B is a table of the MFI of binding to the 25-41 flagellin peptides on the flagellin peptide microarray. FIG. 10C shows binding of mAb F6 to all Lachnospiraceae flagellins on the protein array but not to proteobacteria flagellins.

### Antibody Engineering

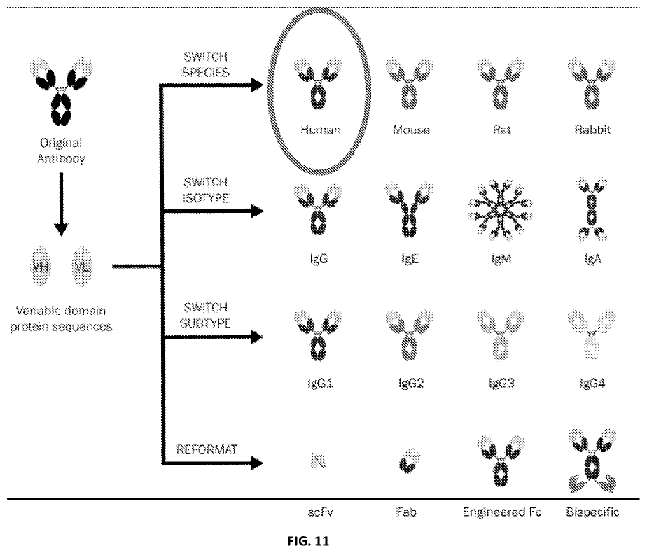

The mAB F6 was further engineered to produce a standardized antibody for use in the epitope assay by using know dilutions of the standardized antibody (e.g., 700-375,000 μg/ml) to produce a standard curve for quantification of IgG responses in a biological sample. The variable region of mAb F6 was combined with the remainder of a human IgG1, as shown in FIG. 11 to produce a chimeric antibody. The amino acid sequences of the heavy chain (SEQ ID NO: 10) and light chain (SEQ ID NO: 11) for the human IgG1 chimeric F6 antibody as well as the nucleic acids encoding the heavy chain (SEQ ID NO: 12) and the amino acid sequence encoding the light chain (SEQ ID NO: 13) are shown below. Amino acid sequences of CDR1-3 in the heavy chain are GYTFTDYY (SEQ ID NO: 14), INPYNGVK (SEQ ID NO: 15), and AWDDGYYGNY (SEQ ID NO: 16), respectively, and the amino acid sequences of CDR1-3 in the light chain are QSLLDSDGKTY (SEQ ID NO: 17), LVS, and WQGTHFPHT (SEQ ID NO: 18), respectively.

Heavy chain
(SEQ ID NO: 10)
EVQLQQSGPVLVKPGASVKMSCKASGYTFTDYYMNWVIQSQGKRL
EWIGVINPYNGVKIYNQKFKGKATLTVDKSSSTAYMELNSLTSED
SAVYYCAWDDGYYGNYWGQGTTLTVSSASTKGPSVFPLAPSSKST
SGGTAALGCLVKDYFPEPVTVSWNSGALTSGVHTFPAVLQSSGLY
SLSSVVTVPSSSLGTQTYICNVNHKPSNTKVDKKVEPKSCDKTHT
CPPCPAPELLGGPSVFLFPPKPKDTLMISRTPEVTCVVVDVSHED
PEVKFNWYVDGVEVHNAKTKPREEQYNSTYRVVSVLTVLHQDWLN
GKEYKCKVSNKALPAPIEKTISKAKGQPREPQVYTLPPSRDELTK
NQVSLTCLVKGFYPSDIAVEWESNGQPENNYKTTPPVLDSDGSFF
LYSKLTVDKSRWQQGNVFSCSVMHEALHNHYTQKSLSLSPGK
Light chain
(SEQ ID NO: 11)
DIVMTQTPLTLSVTIGQPASISCKSSQSLLDSDGKTYLNWLLQRP
GQSPKRLIYLVSKLDSGVPDRFTGSGSGTDFTLKISRMEAEDLGV
YYCWQGTHFPHTFGGGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC
Heavy chain
(SEQ ID NO: 12)
GAGGTCCAACTCCAACAGTCTGGACCTGTGCTGGTGAAGCCTGGA
GCCTCTGTGAAGATGAGTTGTAAGGCATCTGGCTACACCTTCACA
GACTACTATATGAACTGGGTGATTCAGAGCCAGGGCAAGAGATTG
GAGTGGATTGGAGTGATAAACCCATACAATGGAGTGAAGATTTAC
AACCAGAAGTTCAAGGGCAAGGCTACCCTGACAGTGGACAAGTCC
TCCAGCACAGCCTATATGGAACTGAACTCCCTGACCTCTGAGGAC
TCTGCTGTCTACTACTGTGCCTGGGATGATGGCTACTATGGCAAC
TACTGGGGACAAGGCACCACCCTGACAGTGTCCAGTGCTAGCACC
AAGGGCCCATCGGTCTTCCCCCTGGCACCCTCCTCCAAGAGCACC
TCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTAC
TCCCTCAGCAGCGTGGTGACCGTGCCCTCCAGCAGCTTGGGCACC
CAGACCTACATCTGCAACGTGAATCACAAGCCCAGCAACACCAAG
GTGGACAAGAAAGTTGAGCCCAAATCTTGTGACAAAACTCACACA
TGCCCACCGTGCCCAGCACCTGAACTCCTGGGGGGACCGTCAGTC
TTCCTCTTCCCCCCAAAACCCAAGGACACCCTCATGATCTCCCGG
ACCCCTGAGGTCACGTGCGTGGTGGTGGACGTGAGCCACGAAGAC
CCCGAGGTCAAGTTCAACTGGTACGTGGACGGCGTGGAGGTGCAT
AATGCCAAGACAAAGCCGCGGGAGGAGCAGTACAACAGCACGTAC
CGTGTGGTCAGCGTCCTCACCGTCCTGCACCAGGACTGGCTGAAT
GGCAAGGAGTACAAGTGCAAGGTCTCCAACAAAGCCCTCCCAGCC
CCCATCGAGAAAACCATCTCCAAAGCCAAAGGGCAGCCCCGAGAA
CCACAGGTGTACACCCTGCCCCCATCCCGGGATGAGCTGACCAAG
AACCAGGTCAGCCTGACCTGCCTGGTCAAAGGCTTCTATCCCAGC
GACATCGCCGTGGAGTGGGAGAGCAATGGGCAGCCGGAGAACAAC
TACAAGACCACGCCTCCCGTGCTGGACTCCGACGGCTCCTTCTTC
CTCTACAGCAAGCTCACCGTGGACAAGAGCAGGTGGCAGCAGGGG
AACGTCTTCTCATGCTCCGTGATGCATGAGGCTCTGCACAACCAC
TACACGCAGAAGAGCCTCTCCCTGTCTCCGGGTAAATGA
Light chain
(SEQ ID NO: 13)
GACATTGTGATGACCCAGACACCACTGACCCTGTCTGTGACCATT
GGACAACCTGCCAGCATCTCCTGTAAGTCCAGCCAGTCCCTGCTG
GACTCTGATGGCAAGACCTACCTGAACTGGCTGCTCCAAAGACCT
GGACAAAGCCCAAAGAGACTGATTTACCTGGTGAGCAAACTGGAC
TCTGGAGTGCCTGACAGGTTCACAGGCTCTGGCTCTGGCACAGAC
TTCACCCTGAAAATCAGCAGGATGGAGGCTGAGGACCTGGGAGTC
TACTACTGTTGGCAGGGCACCCACTTTCCACACACCTTTGGAGGA
GGCACCAAATTGGAGATTAAGCGTACGGTGGCTGCACCATCTGTC
TTCATCTTCCCGCCATCTGATGAGCAGTTGAAATCTGGAACTGCC
TCTGTTGTGTGCCTGCTGAATAACTTCTATCCCAGAGAGGCCAAA
GTACAGTGGAAGGTGGATAACGCCCTCCAATCGGGTAACTCCCAG
GAGAGTGTCACAGAGCAGGACAGCAAGGACAGCACCTACAGCCTC
AGCAGCACCCTGACGCTGAGCAAAGCAGACTACGAGAAACACAAA
GTCTACGCCTGCGAAGTCACCCATCAGGGCCTGAGCTCGCCCGTC
ACAAAGAGCTTCAACAGGGGAGAGTGTTAG

A murine mAB F6 (F6 IgG2b) monoclonal antibody was produced as described above. The human IgG1 F6 chimeric antibody has the same binding region as the murine F6 IgG2B antibody. The murine mAB F6 antibody comprises a heavy chain comprising the amino acid sequence set forth as SEQ ID NO: 28 and a light chain comprising the amino acid sequence set forth as SEQ ID NO: 29. The nucleic acid sequence encoding the heavy chain comprises SEQ ID NO: 30 and the nucleic acid sequence encoding the light chain comprises SEQ ID NO: 31. The amino acid sequences of the heavy chain (SEQ ID NO: 28) and light chain (SEQ ID NO: 29) for the murine IgG1 F6 antibody as well as the nucleic acids encoding the heavy chain (SEQ ID NO: 30) and the amino acid sequence encoding the light chain (SEQ ID NO: 31) are shown below. Amino acid sequences of CDR1-3 in the heavy chain are GYTFTDYY (SEQ ID NO: 14), INPYNGVK (SEQ ID NO: 15), and AWDDGYYGNY (SEQ ID NO: 16), respectively, and the amino acid sequences of CDR1-3 in the light chain are QSLLDSDGKTY (SEQ ID NO: 17), LVS, and WQGTHFPHT (SEQ ID NO: 18), respectively.

Heavy chain
(SEQ ID NO: 28)
EVQLQQSGPVLVKPGASVKMSCKASGYTFTDYYMNWVIQSQGKRL
EWIGVINPYNGVKIYNQKFKGKATLTVDKSSSTAYMELNSLTSED
SAVYYCAWDDGYYGNYWGQGTTLTVSSAKTTPPSVYPLAPGCGDT
TGSSVTLGCLVKGYFPESVTVTWNSGSLSSSVHTFPALLQSGLYT
MSSSVTVPSSTWPSQTVTCSVAHPASSTTVDKKLEPSGPISTINP
CPPCKECHKCPAPNLEGGPSVFIFPPNIKDVLMISLTPKVTCVVV
DVSEDDPDVQISWFVNNVEVHTAQTQTHREDYNSTIRVVSTLPIQ
HQDWMSGKEFKCKVNNKDLPSPIERTISKIKGLVRAPQVYILPPP
AEQLSRKDVSLTCLVVGFNPGDISVEWTSNGHTEENYKDTAPVLD
SDGSYFIYSKLNMKTSKWEKTDSFSCNVRHEGLKNYYLKKTISRS
PGK
Light chain
(SEQ ID NO: 29)
DIVMTQTPLTLSVTIGQPASISCKSSQSLLDSDGKTYLNWLLQRP
GQSPKRLIYLVSKLDSGVPDRFTGSGSGTDFTLKISRMEAEDLGV
YYCWQGTHFPHTFGGGTKLEIKRADAAPTVSIFPPSSEQLTSGGA
SVVCFLNNFYPKDINVKWKIDGSERQNGVLNSWTDQDSKDSTYSM
SSTLTLTKDEYERHNSYTCEATHKTSTSPIVKSFNRNEC
Heavy chain
(SEQ ID NO: 30)
GAGGTCCAACTCCAACAGTCTGGACCTGTGCTGGTGAAGCCTGGA
GCCTCTGTGAAGATGAGTTGTAAGGCATCTGGCTACACCTTCACA
GACTACTATATGAACTGGGTGATTCAGAGCCAGGGCAAGAGATTG
GAGTGGATTGGAGTGATAAACCCATACAATGGAGTGAAGATTTAC
AACCAGAAGTTCAAGGGCAAGGCTACCCTGACAGTGGACAAGTCC
TCCAGCACAGCCTATATGGAACTGAACTCCCTGACCTCTGAGGAC
TCTGCTGTCTACTACTGTGCCTGGGATGATGGCTACTATGGCAAC
TACTGGGGACAAGGCACCACCCTGACAGTGTCCAGTGCCAAAACA
ACACCCCCATCAGTCTATCCACTGGCCCCTGGGTGTGGAGATACA
ACTGGTTCCTCCGTGACTCTGGGATGCCTGGTCAAGGGCTACTTC
CCTGAGTCAGTGACTGTGACTTGGAACTCTGGATCCCTGTCCAGC
AGTGTGCACACCTTCCCAGCTCTCCTGCAGTCTGGACTCTACACT
ATGAGCAGCTCAGTGACTGTCCCCTCCAGCACCTGGCCAAGTCAG
ACCGTCACCTGCAGCGTTGCTCACCCAGCCAGCAGCACCACGGTG
GACAAAAAACTTGAGCCCAGCGGGCCCATTTCAACAATCAACCCC
TGTCCTCCATGCAAGGAGTGTCACAAATGCCCAGCTCCTAACCTC
GAGGGTGGACCATCCGTCTTCATCTTCCCTCCAAATATCAAGGAT
GTACTCATGATCTCCCTGACACCCAAGGTCACGTGTGTGGTGGTG
GATGTGAGCGAGGATGACCCAGACGTCCAGATCAGCTGGTTTGTG
AACAACGTGGAAGTACACACAGCTCAGACACAAACCCATAGAGAG
GATTACAACAGTACTATCCGGGTGGTCAGCACCCTCCCCATCCAG
CACCAGGACTGGATGAGTGGCAAGGAGTTCAAATGCAAGGTCAAC
AACAAAGACCTCCCATCACCCATCGAGAGAACCATCTCAAAAATT
AAAGGGCTAGTCAGAGCTCCACAAGTATACATCTTGCCGCCACCA
GCAGAGCAGTTGTCCAGGAAAGATGTCAGTCTCACTTGCCTGGTC
GTGGGCTTCAACCCTGGAGACATCAGTGTGGAGTGGACCAGCAAT
GGGCATACAGAGGAGAACTACAAGGACACCGCACCAGTCCTGGAC
TCTGACGGTTCTTACTTCATATATAGCAAGCTCAATATGAAAACA
AGCAAGTGGGAGAAAACAGATTCCTTCTCATGCAACGTGAGACAC
GAGGGTCTGAAAAATTACTACCTGAAGAAGACCATCTCCCGGTCT
CCGGGTAAATGA
Light chain
(SEQ ID NO: 31)
GACATTGTGATGACCCAGACACCACTGACCCTGTCTGTGACCATT
GGACAACCTGCCAGCATCTCCTGTAAGTCCAGCCAGTCCCTGCTG
GACTCTGATGGCAAGACCTACCTGAACTGGCTGCTCCAAAGACCT
GGACAAAGCCCAAAGAGACTGATTTACCTGGTGAGCAAACTGGAC
TCTGGAGTGCCTGACAGGTTCACAGGCTCTGGCTCTGGCACAGAC
TTCACCCTGAAAATCAGCAGGATGGAGGCTGAGGACCTGGGAGTC
TACTACTGTTGGCAGGGCACCCACTTTCCACACACCTTTGGAGGA
GGCACCAAATTGGAGATTAAGCGGGCTGATGCTGCACCAACTGTA
TCCATCTTCCCACCATCCAGTGAGCAGTTAACATCTGGAGGTGCC
TCAGTCGTGTGCTTCTTGAACAACTTCTACCCCAAAGACATCAAT
GTCAAGTGGAAGATTGATGGCAGTGAACGACAAAATGGCGTCCTG
AACAGTTGGACTGATCAGGACAGCAAAGACAGCACCTACAGCATG
AGCAGCACCCTCACGTTGACCAAGGACGAGTATGAACGACATAAC
AGCTATACCTGTGAGGCCACTCACAAGACATCAACTTCACCCATT
GTCAAGAGCTTCAACAGGAATGAGTGTTAA

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to one or more molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

Articles “a” and “an” are used herein to refer to one or to more than one (i.e. at least one) of the grammatical object of the article. By way of example, “an element” means at least one element and can include more than one element.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

The terms “optional” and “optionally” mean that the subsequently described event, circumstance, or material may or may not occur or be present, and that the description includes instances where the event, circumstance, or material occurs or is present as well as instances where it does not occur or is not present.

1. A peptide array comprising a plurality of Lachnospiraceae flagellin peptides, wherein the plurality of Lachnospiraceae flagellin peptides are fragments of a hinge region of a Lachnospiraceae flagellin and wherein the plurality of Lachnospiraceae flagellin peptides are attached to a substrate.

2. The peptide array of claim 1, wherein the plurality of Lachnospiraceae flagellin peptides comprise the amino acid sequences of SEQ ID NO: 3, SEQ ID NO: 4, and SEQ ID NO: 5.

3. The peptide array of claim 2, wherein the plurality of Lachnospiraceae flagellin peptides further comprise the amino acid sequences of SEQ ID NO: 2, SEQ ID NO: 6, SEQ ID NO: 7, SEQ ID NO: 8, and SEQ ID NO: 9.

4. The peptide array of claim 1, wherein the substrate is a solid support.

5. The peptide array of claim 1, wherein the substrate is a moveable substrate.

6. The peptide array of claim 4, wherein the moveable substrate comprises beads.

7. The peptide array of claim 6, wherein each different Lachnospiraceae flagellin peptide of the plurality is attached to a different type of bead.

8. The peptide array of claim 7, wherein each type of bead has a different size, a different detectable label, or both a different size and a different detectable label.

9. The peptide array of claim 8, wherein the detectable label is a fluorophore.

10. An antibody or fragment thereof that binds to one or more of the plurality of flagellin peptides in the peptide array of claim 1, wherein the antibody or fragment thereof comprises CDR amino acid sequences having at least 90 percent identity to GYTFTDYY (SEQ ID NO: 14), INPYNGVK (SEQ ID NO: 15), and AWDDGYYGNY (SEQ ID NO: 16), QSLLDSDGKTY (SEQ ID NO: 17), LVS, and WQGTHFPHT (SEQ ID NO: 18).

11. The antibody or fragment thereof of claim 10, wherein the antibody or fragment thereof comprises CDR amino acid sequences of GYTFTDYY (SEQ ID NO: 14), INPYNGVK (SEQ ID NO: 15), and AWDDGYYGNY (SEQ ID NO: 16), QSLLDSDGKTY (SEQ ID NO: 17), LVS, and WQGTHFPHT (SEQ ID NO: 18).

12. The antibody or fragment thereof of claim 11, wherein the antibody or fragment thereof comprises a heavy chain amino acid sequence of SEQ ID NO: 10 and a light chain amino acid sequence of SEQ ID NO: 11.

13. A method of identifying an immunosignature in a subject with or suspected of having an immune-mediated disease or in a subject at risk of an immune-mediated disease comprising
(a) contacting a biological sample from the subject with the peptide array of claim 1 under conditions that allow binding of antibodies in the biological sample with the plurality of Lachnospiraceae peptides in the peptide array and
(b) detecting the level of binding of antibodies in the biological sample with the plurality of Lachnospiraceae peptides in the peptide array to form an immunosignature for the subject.

14. The method of claim 13, wherein detecting the level of bound antibodies comprises detection of bound immunoglobulin G.

15. The method of claim 13, further comprising comparing the subject's immunosignature with an immunosignature of one or more control subjects, wherein the one or more control subjects are known to be either negative for immune-mediated diseases or positive for a specific immune-mediated disease.

16. The method of claim 13, further comprising detecting the level of binding of the antibodies in the biological sample with the plurality of flagellin peptides in the peptide array using one or more amounts of a control antibody or fragment thereof that binds to one or more of plurality of the flagellin peptides.

17. The method of claim 16, wherein the control antibody or fragment thereof comprises CDR amino acid sequences having at least 90 percent identity to GYTFTDYY (SEQ ID NO: 14), INPYNGVK (SEQ ID NO: 15), and AWDDGYYGNY (SEQ ID NO: 16), QSLLDSDGKTY (SEQ ID NO: 17), LVS, and WQGTHFPHT (SEQ ID NO: 18).

18. The method of claim 17, wherein the control antibody or fragment thereof comprises CDR amino acid sequences of GYTFTDYY (SEQ ID NO: 14), INPYNGVK (SEQ ID NO: 15), and AWDDGYYGNY (SEQ ID NO: 16), QSLLDSDGKTY (SEQ ID NO: 17), LVS, and WQGTHFPHT (SEQ ID NO: 18).

19. The method of claim 18, wherein the control antibody or fragment thereof comprises a heavy chain amino acid sequence of SEQ ID NO: 10 and a light chain amino acid sequence of SEQ ID NO: 11.

20. The method of claim 18, wherein the control antibody or fragment thereof comprises a heavy chain amino acid sequence of SEQ ID NO: 28 and a light chain amino acid sequence of SEQ ID NO: 29.

21. A method for treating an immune-mediated disease in a subject comprising:
(a) identifying an immunosignature in a subject with or suspected of having an immune-mediated disease or in a subject at risk of an immune-mediated disease contacting a biological sample from a subject with the peptide array of claim 1;
(b) associating the immunosignature with an immune-mediated disease in the subject, and
(c) administering a therapeutic agent to the subject having immune-mediated disease.

22. The method of claim 20, wherein the immune-mediated disease is an inflammatory bowel disease.

23. The method of claim 22, wherein the inflammatory bowel disease is Crohn's disease.

24. The method of claim 20, wherein the immunosignature is associated with an immune-mediated disease responsive to an anti-flagellin therapeutic agent.

25. The method of claim 24, wherein the therapeutic agent is a polypeptide comprising one of more flagellin T cell receptor epitopes.

26. A method for treating an immune-mediated disease in a subject comprising:
(a) obtaining a first biological sample from the subject prior to administration of a therapeutic agent;
(b) identifying a first immunosignature for a subject by contacting the first biological sample from the subject with the peptide array of claim 1;
(c) administering to the subject an effective amount of the therapeutic agent;
(d) obtaining a second biological sample from the subject following administration of the therapeutic agent;
(e) identifying a second immunosignature for the subject by contacting the second biological sample from the subject with the peptide array of any one of claim 1;
(f) comparing the first and second immunosignatures; and
(g) administering the therapeutic agent to the subject subsequent to the comparing step (f) if the second immunosignature is more similar to the immunosignature of one or more control subjects known to negative for immune-mediated diseases as compared to the first immunosignature; or
(h) modifying the dosage regimen of the therapeutic agent or administering a different therapeutic agent to the subject subsequent to the comparing step (f) if the second immunosignature is more similar to the immunosignature of one or more control subjects known to be positive for immune-mediated diseases, as compared to the first immunosignature.

27. The method of claim 26, wherein the immune-mediated disease is an inflammatory bowel disease.

28. The method of claim 27, wherein the inflammatory bowel disease is Crohn's disease.

29. The method of claim 26, wherein the therapeutic agent is a polypeptide comprising one of more flagellin T cell receptor epitopes.

30. A flagellin-binding monoclonal antibody comprising a variable heavy (VH) chain comprising SEQ ID NO: 10 and a variable light (VL) chain comprising SEQ ID NO: 11.

31. A flagellin-binding monoclonal antibody comprising a variable heavy (VH) chain comprising SEQ ID NO: 28 and a variable light (VL) chain comprising SEQ ID NO: 29.

32. The flagellin-binding monoclonal antibody of claim 30, wherein the antibody comprises CDR amino acid sequences of GYTFTDYY (SEQ ID NO: 14), INPYNGVK (SEQ ID NO: 15), and AWDDGYYGNY (SEQ ID NO: 16), QSLLDSDGKTY (SEQ ID NO: 17), LVS, and WQGTHFPHT (SEQ ID NO: 18).
